SEQUENCE LISTING (22 sequences)

SEQ ID NO: 1 - protein, 5 aa, synthetic construct
  REGION 1..5: note = HCDR1
SYYMS

SEQ ID NO: 2 - protein, 17 aa, synthetic construct
  REGION 1..17: note = HCDR2
TIIKSGGYAYYPDSVKD

SEQ ID NO: 3 - protein, 8 aa, synthetic construct
  REGION 1..8: note = HCDR3
GGQGAMDY

SEQ ID NO: 4 - protein, 11 aa, synthetic construct
  REGION 1..11: note = LCDR1
RASQSIGDYLH

SEQ ID NO: 5 - protein, 7 aa, synthetic construct
  REGION 1..7: note = LCDR2
YASQSIS

SEQ ID NO: 6 - protein, 9 aa, synthetic construct
  REGION 1..9: note = LCDR3
QQGYSFPYT

SEQ ID NO: 7 - protein, 117 aa, synthetic construct
  REGION 1..117: note = VH - variant 1
EVQLVESGGGLVQPGGSLRLSCAASGFTFSSYYMSWVRQAPGKGLEWVSTIIKSGGYAYYPDSVKDRFTISRDNSKNTLYLQMSSLRAEDTAVYYCVRGGQGAMDYWGQGTTVTVSS

SEQ ID NO: 8 - protein, 117 aa, synthetic construct
  REGION 1..117: note = VH - variant 2
EVQLVESGGGLVQPGGSLRLSCAASGFPFSSYYMSWVRQAPGKGLEWVSTIIKSGGYAYYPDSVKDRFTISRDNSKNTLYLQMSSLRAEDTAVYYCVRGGQGAMDYWGQGTTVTVSS

SEQ ID NO: 9 - protein, 117 aa, synthetic construct
  REGION 1..117: note = VH - variant 3
EVQLVESGGGLVQLGGSLRLSCAASGFTFSSYYMSWVRQAPGKGLEWVSTIIKSGGYAYYPDSVKDRFTISRDNSKNTLYLQMNSLRAEDTAVYYCVKGGQGAMDYWGQGTTVTVSS

SEQ ID NO: 10 - protein, 117 aa, synthetic construct
  REGION 1..117: note = VH- variant 4
EVQLVESGGGLLQLGGSLRLSCAASGFTFSSYYMSWVRQAPGKGLEWVSTIIKSGGYAYYPDSVKDRFTISRDNSKNTLNLQMNSLRAEDTAVYYCVKGGQGAMDYWGQGTTVTVSS

SEQ ID NO: 11 - protein, 107 aa, synthetic construct
  REGION 1..107: note = VL - variant a
EIVLTQSPATLSLSPGERATLSCRASQSIGDYLHWYQQKPGQAPRLLIKYASQSISGIPARFSGSGSGTDFTLTITSLEPEDFAVYYCQQGYSFPYTFGGGTKLEIK

SEQ ID NO: 12 - protein, 107 aa, synthetic construct
  REGION 1..107: note = VL - variant b
EIVLTQSPATLSLSPGERATLSCRASQSIGDYLHWYQQKPGQAPRLLIYYASQSISGIPARFSGSGSGTDFTLTISSLEPEDFAVYYCQQGYSFPYTFGGGTKLEIK

SEQ ID NO: 13 - protein, 107 aa, synthetic construct
  REGION 1..107: note = VL- variant c
EIVLTQSPATLSLSPGERATLSCRASQSIGDYLHWYQQKPGQAPRLLIKYASQSISGIPARFSGSGSGTDFTLTISSLEPEDFAVYYCQQGYSFPYTFGGGTKLEIK

SEQ ID NO: 14 - protein, 446 aa, synthetic construct
  REGION 1..446: note = Heavy Chain- Clone I
EVQLVESGGGLVQPGGSLRLSCAASGFTFSSYYMSWVRQAPGKGLEWVSTIIKSGGYAYYPDSVKDRFTISRDNSKNTLYLQMSSLRAEDTAVYYCVRGGQGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 214 aa, synthetic construct
  REGION 1..214: note = Light Chain - Clone I
EIVLTQSPATLSLSPGERATLSCRASQSIGDYLHWYQQKPGQAPRLLIKYASQSISGIPARFSGSGSGTDFTLTITSLEPEDFAVYYCQQGYSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 446 aa, synthetic construct
  REGION 1..446: note = Heavy Chain - Clone II
EVQLVESGGGLVQPGGSLRLSCAASGFPFSSYYMSWVRQAPGKGLEWVSTIIKSGGYAYYPDSVKDRFTISRDNSKNTLYLQMSSLRAEDTAVYYCVRGGQGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 446 aa, synthetic construct
  REGION 1..446: note = Heavy Chain - Clone III
EVQLVESGGGLVQLGGSLRLSCAASGFTFSSYYMSWVRQAPGKGLEWVSTIIKSGGYAYYPDSVKDRFTISRDNSKNTLYLQMNSLRAEDTAVYYCVKGGQGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 214 aa, synthetic construct
  REGION 1..214: note = Light Chain - Clone III
EIVLTQSPATLSLSPGERATLSCRASQSIGDYLHWYQQKPGQAPRLLIYYASQSISGIPARFSGSGSGTDFTLTISSLEPEDFAVYYCQQGYSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 446 aa, synthetic construct
  REGION 1..446: note = Heavy Chain- Clone IV
EVQLVESGGGLLQLGGSLRLSCAASGFTFSSYYMSWVRQAPGKGLEWVSTIIKSGGYAYYPDSVKDRFTISRDNSKNTLNLQMNSLRAEDTAVYYCVKGGQGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 214 aa, synthetic construct
  REGION 1..214: note = Light Chain - Clone IV
EIVLTQSPATLSLSPGERATLSCRASQSIGDYLHWYQQKPGQAPRLLIKYASQSISGIPARFSGSGSGTDFTLTISSLEPEDFAVYYCQQGYSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 13 aa, synthetic construct
  REGION 1..13: note = Epitope
DSTKDLPDDVITF

SEQ ID NO: 22 - protein, 751 aa, Homo sapiens
NYQNGKNNVPRLKLSYKEMLESNNVITFNGLANSSSYHTFLLDEERSRLYVGAKDHIFSFDLVNIKDFQKIVWPVSYTRRDECKWAGKDILKECANFIKVLKAYNQTHLYACGTGAFHPICTYIEIGHHPEDNIFKLENSHFENGRGKSPYDPKLLTASLLIDGELYSGTAADFMGRDFAIFRTLGHHHPIRTEQHDSRWLNDPKFISAHLISESDNPEDDKVYFFFRENAIDGEHSGKATHARIGQICKNDFGGHRSLVNKWTTFLKARLICSVPGPNGIDTHFDELQDVFLMNFKDPKNPVVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPMNNRPIVIKTDVNYQFTQIVVDRVDAEDGQYDVMFIGTDVGTVLKVVSIPKETWYDLEEVLLEEMTVFREPTAISAMELSTKQQQLYIGSTAGVAQLPLHRCDIYGKACAECCLARDPYCAWDGSACSRYFPTAKRRTRRQDIRNGDPLTHCSDLHHDNHHGHSPEERIIYGVENSSTFLECSPKSQRALVYWQFQRRNEERKEEIRVDDHIIRTDQGLLLRSLQQKDSGNYLCHAVEHGFIQTLLKVTLEVIDTEHLEELLHKDDDGDGSKTKEMSNSMTPSQKVWYRDFMQLINHPNLNTMDEFCEQVWKRDRKQRRQRPGHTPGNSNKWKHLQENKKGRNRRTHEFERAPRSV

(19) United States
(12) Patent Application Publication
(10) Pub. No.: US 2024/0228600 A1
(43) Pub. Date: Jul. 11, 2024

(54) ANTI-SEMA3A ANTIBODIES AND THEIR USES FOR TREATING A THROMBOTIC DISEASE OF THE RETINA

(71) Applicant: Boehringer Ingelheim International GmbH, Ingelheim am Rhein (DE)

(72) Inventors: Leo THOMAS, Biberach an der Riss (DE); Remko Alexander BAKKER, Biberach an der Riss (DE)

(21) Appl. No.: 18/533,296

(22) Filed: Dec. 8, 2023

Related U.S. Application Data
(63) Continuation of application No. 17/506,667, filed on Oct. 21, 2021.

(30) Foreign Application Priority Data
Oct. 23, 2020 (EP) 20203645.5

(51) Int. Cl. C07K 16/18; A61K 39/00; A61P 7/02

(52) U.S. Cl. CPC C07K 16/18; A61P 7/02; A61K 2039/505; C07K 2317/51; C07K 2317/92

(57) ABSTRACT

The invention relates to the use of antibodies and antibody that target semaphorin 3A (Sema3A), and fragments thereof, and their use for treating thrombotic diseases of the retina comprising.

### RELATED APPLICATION DISCLOSURE

This application is a continuation of U.S. application Ser. No. 17/506,667 filed Oct. 21, 2021, which claims the benefit of European Patent Application No. 20203645.5 filed Oct. 23, 2020, each of which is hereby incorporated by reference herein in its entirety.

### SEQUENCE LISTING

This application includes, as part of its disclosure, a “Sequence Listing XML” pursuant to 37 C.F.R. § 1.831(a) which is submitted in XML file format via the USPTO patent electronic filing system in a file named “01-3450-US-2.xml”, created on Nov. 28, 2023, and having a size of 31,632 bytes, which is hereby incorporated by reference herein in its entirety.

### FIELD OF THE INVENTION

This invention generally relates to antibodies and fragments thereof that target semaphorin 3A (Sema3A) for use for treating a thrombotic disease of the retina.

### BACKGROUND OF THE INVENTION

Retinal vein occlusion (RVO) is a restriction or blockage of blood flow leaving the retina and is the second most common retinal vascular disorder after diabetic retinopathy. Causing varying degrees of vision loss, central retinal vein occlusion (CRVO), and branch retinal vein occlusion (BRVO) can be complicated by macular edema that can lead to total blindness.

No treatment is available to reverse retinal vein occlusions. However, the iris or retinal neovascularization or macular edema may be managed with anti-VEGF or steroid injections. Other therapeutic approaches include use of laser and surgery. However, none of the existing therapeutic approaches prove a reliable, safe and successful outcome for patients suffering from RVO. Consequently, there is still an unfulfilled need for new therapeutic approaches for efficiently treating thrombotic diseases of the retina.

### SUMMARY OF THE INVENTION

Sema3A is an endogenous secreted protein that belongs to the class 3 semaphorin family (Sema3), which were originally identified as axonal guidance molecules and were implicated in vessel pathfinding and network formation. Neuropilin 1 and 2 (Nrp1 and Nrp2) and the type A/D plexins (Plxns) act as the ligands binding and the signal transducing subunits of the Sema3 receptor complexes on the surface of endothelial cells (ECs). As a special member of the Sema3 family, Sema3A binds to Nrp1 exclusively at first and then combines with PlexinA1-4 as a complex (Nrp1/PlexA1-4). In this receptor complex, Nrp1 acts as a binding element, while PlexA1-4 acts as a signal-transducing element.

Human semaphorin 3A is a protein as disclosed in SEQ ID NO: 22 and available under the NCBI Reference Sequence NP_006071.1. Further, human Sema3A is encoded by the Gene ID: 10371 (NCBI).

Sema3A has been studied in tumor angiogenesis and metastasis for years, but its effects on retinal neovascularization are still unclear. The inventors have exemplified that Semaphorin 3A is secreted by hypoxic retinal ganglion cells and acts as a vasorepulsive cue. Sema3A repels neovessels away from ischemic region by inducing a cytoskeletal collapse in these cells. Without wishing to be bound by theory, the inventors have hypothesized that this would explain why revascularization of ischemic regions does not occur and instead the up-regulation of Sema3A leads to a pathological neovascularisation into the vitreous region.

Semaphorin 3A is secreted by hypoxic neurons in ischemic/avascular retina, thereby inhibiting vascular regeneration of the retina and enhancing pathologic preretinal neovascularization.

The inventors have harnessed their understanding of the Sema3A biology and impact on the retina for developing a new therapeutic strategy for treating thrombotic diseases of the retina. Thus, in a first aspect, the present invention provides an anti-Sema3A antibody or an antigen-binding fragment thereof for use for treating a thrombotic disease of the retina, said anti-Sema3A antibody or antigen-binding fragment thereof comprising:
a heavy chain variable region comprising the amino acid sequence of SEQ ID NO: 1 (H-CDR1); the amino acid sequence of SEQ ID NO: 2 (H-CDR2); and the amino acid sequence of SEQ ID NO: 3 (H-CDR3); and
a light chain variable region comprising the amino acid sequence of SEQ ID NO: 4 (L-CDR1); the amino acid sequence of SEQ ID NO: 5 (L-CDR2); and the amino acid sequence of SEQ ID NO: 6 (L-CDR3).

In one embodiment, said anti-Sema3A antibody or antigen-binding fragment thereof comprises:
a heavy chain variable region comprising an amino acid sequence at least 80%, at least 90%, at least 95%, at least 98%, or at least 99% identical to the amino acid sequence SEQ ID NO: 7, SEQ ID NO: 8, SEQ ID NO: 9 or SEQ ID NO: 10; and
a light chain variable region comprising an amino acid sequence at least 80%, at least 90%, at least 95%, at least 98%, or at least 99% identical to the amino acid sequence of SEQ ID NO: 11, SEQ ID NO: 12 or SEQ ID NO: 13;
   wherein:
the heavy chain variable region comprises the amino acid sequence of SEQ ID NO: 1 (H-CDR1); the amino acid sequence of SEQ ID NO: 2 (H-CDR2); and the amino acid sequence of SEQ ID NO: 3 (H-CDR3); and
the light chain variable region comprises the amino acid sequence of SEQ ID NO: 4 (L-CDR1); the amino acid sequence of SEQ ID NO: 5 (L-CDR2); and the amino acid sequence of SEQ ID NO: 6 (L-CDR3).

In another embodiment, said anti-Sema3A antibody or an antigen-binding fragment thereof comprises:
a heavy chain variable region comprising an amino acid sequence at least 80%, at least 90%, at least 95%, at least 98%, or at least 99% identical to the amino acid sequence SEQ ID NO: 7, SEQ ID NO: 8, SEQ ID NO: 9 or SEQ ID NO: 10; and
a light chain variable region comprising an amino acid sequence at least 80%, at least 90%, at least 95%, at least 98%, or at least 99% identical to the amino acid sequence of SEQ ID NO: 11, SEQ ID NO: 12 or SEQ ID NO: 13.

In yet another embodiment, said anti-Sema3A antibody or antigen-binding fragment thereof comprises:
a heavy chain variable region comprising the amino acid sequences of SEQ ID NO: 7, SEQ ID NO: 8, SEQ ID NO: 9 or SEQ ID NO: 10; and
a light chain variable region comprising the amino acid sequence of SEQ ID NO: 11, SEQ ID NO: 12 or SEQ ID NO: 13.

In another embodiment, said anti-Sema3A antibody or antigen-binding fragment thereof comprises:
a. a variable heavy chain and a variable light chain comprising the amino acid sequences of SEQ ID NO: 7 and SEQ ID NO: 11, respectively;
b. a variable heavy chain and a variable light chain comprising the amino acid sequences of SEQ ID NO: 8 and SEQ ID NO: 11, respectively;
c. a variable heavy chain and a variable light chain comprising the amino acid sequences of SEQ ID NO: 9 and SEQ ID NO: 12, respectively; or
d. a variable heavy chain and a variable light chain comprising the amino acid sequences of SEQ ID NO: 10 and SEQ ID NO: 13, respectively.

In yet another embodiment, said anti-Sema3A antibody or antigen-binding fragment thereof comprises:
a heavy chain comprising, preferably consisting of, the amino acid sequence of SEQ ID NO: 14, SEQ ID NO: 16, SEQ ID NO: 17, or SEQ ID NO: 19; and
a light chain comprising, preferably consisting of, the amino acid sequence of SEQ ID NO: 15, SEQ ID NO: 18 or SEQ ID NO: 20.

In a particular embodiment, said anti-Sema3A antibody or antigen-binding fragment thereof comprises:
a. a heavy chain comprising the amino acid sequence of SEQ ID NO: 14 and a light chain comprising the amino acid sequence of SEQ ID NO: 15;
b. a heavy chain comprising the amino acid sequence of SEQ ID NO: 16 and a light chain comprising the amino acid sequence of SEQ ID NO: 15;
c. a heavy chain comprising the amino acid sequence of SEQ ID NO: 17 and a light chain comprising the amino acid sequence of SEQ ID NO: 18; or
d. a heavy chain comprising the amino acid sequence of SEQ ID NO: 19 and a light chain comprising the amino acid sequence of SEQ ID NO: 20.

In a particular preferred embodiment, said anti-Sema3A antibody is a humanized anti-Sema3A antibody.

In a preferred embodiment, said thrombotic disease of the retina is selected from the group consisting of retinal vein occlusion (RVO) including central retinal vein occlusion (CRVO), hemispheric retinal vein occlusion (HRVO), branch retinal vein occlusion (BRVO), and arterial occlusive disease of the retina. In a yet preferred embodiment, said thrombotic disease of the retina is selected from the group consisting of retinal vein occlusion (RVO) including central retinal vein occlusion (CRVO), hemispheric retinal vein occlusion (HRVO) and branch retinal vein occlusion (BRVO).

In a second aspect, the present invention provides an anti-Sema3A antibody or an antigen-binding fragment thereof that binds to at least one amino acid residue within amino acid regions 370-382 of the human Sema3A as depicted in SEQ ID NO: 22 for use for treating a thrombotic disease of the retina. Preferably, said thrombotic disease of the retina is selected from the group consisting of retinal vein occlusion (RVO) including central retinal vein occlusion (CRVO), hemispheric retinal vein occlusion (HRVO), branch retinal vein occlusion (BRVO), and arterial occlusive disease of the retina.

In one embodiment, said anti-Sema3A antibody or antigen-binding fragment thereof binds to at least one amino acid residue within amino acid regions as set forth in SEQ ID NO: 21 (DSTKDLPDDVITF). In a preferred embodiment, the present invention provides an anti-Sema3A antibody or an antigen-binding fragment thereof that binds the amino acid regions as set forth in SEQ ID NO: 21 for use for treating a thrombotic disease of the retina.

In one embodiment, the present invention provides an anti-Sema3A or an antigen-binding fragment for use for treating a thrombotic disease of the retina by inhibiting the vasorepressive effect of SemaA, by improving revascularisation of the retina and/or by reducing permeability of blood retinal barrier.

In a preferred embodiment, the present invention provides an anti-Sema3A antibody or an antigen-binding fragment thereof for use for treating a thrombotic disease of the retina in a patient suffering from diabetic macular ischemia, preferably by promoting vascular regeneration within the ischemic retina (revascularization) and preventing pathological neovascularization of the vitreous region of the eye.

In another preferred embodiment, the present invention provides an anti-Sema3A antibody or an antigen-binding fragment thereof for use for treating a thrombotic disease of the retina in a patient suffering from diabetic macular edema, preferably by reducing permeability of blood retinal barrier.

In another preferred embodiment, the present invention provides an anti-Sema3A antibody or an antigen-binding fragment thereof for use for treating a thrombotic disease of the retina, by inhibiting Sema3A-induced permeability of the blood retinal barrier and/or Sema3A-induced vasoregression from ischemic areas.

In a fourth aspect, the present invention provides a pharmaceutical composition comprising an anti-Sema3A antibody or an antigen-binding fragment thereof and a pharmaceutically acceptable carrier for use for treating a thrombotic disease of the retina.

### BRIEF DESCRIPTION OF THE DRAWINGS

FIGS. 1A and 1B depict the protocol of a study directed to the use of the antibody of the invention via intravitreal injection in retinal ischemia using retinal vein occlusion model of mice. The treatment protocol comprises an early phase administration after laser irradiation (FIG. 1A, Study 1) and a late phase administration at 7 days after the laser irradiation (FIG. 1B, Study 2). The anti-Sema3A antibody and/or anti-VEGF trap is intravitreally injected either immediately after laser irradiation (study 1) or 7 days after laser irradiation (FIG. 1B, Study 2) into the eyes of mice.

FIGS. 2A and 2B show the changes in the ocular blood flow with laser speckle flowgraphy at 1 or 8 days after the laser irradiation by the vehicle, the anti-Sema3A antibody of the invention, the VEGF-trap Eylea® or the combination of the anti-Sema3A antibody of the invention and the VEGF-trap Eylea®. FIG. 2A depicts the results in the early phase administration after laser irradiation, FIG. 2B depicts the results in late phase administration after laser irradiation. Data are shown as mean±S.E.M (n=5). ##P<0.01 (versus vehicle-treated group).

### DETAILED DESCRIPTION OF THE INVENTION

### Definitions

The generalized structure of antibodies or immunoglobulin is well known to the person skilled in the art, these molecules are heterotetrameric glycoproteins, typically of about 150,000 daltons, composed of two identical light (L) chains and two identical heavy (H) chains. Each light chain is covalently linked to a heavy chain by one disulfide bond to form a heterodimer, and the heterotrimeric molecule is formed through a covalent disulfide linkage between the two identical heavy chains of the heterodimers. Although the light and heavy chains are linked together by one disulfide bond, the number of disulfide linkages between the two heavy chains varies by immunoglobulin isotype. Each heavy and light chain also has regularly spaced intrachain disulfide bridges. Each heavy chain has at the amino-terminus a variable domain (V_{H}=variable heavy chain), followed by three or four constant domains (C_{H1}, C_{H2}, C_{H3}, and C_{H4}), as well as a hinge region between C_{H1} and C_{H2}. Each light chain has two domains, an amino-terminal variable domain (V_{L}=variable light chain) and a carboxy-terminal constant domain (C_{L}). The V_{L} domain associates non-covalently with the V_{H} domain, whereas the C_{L} domain is commonly covalently linked to the C_{H1} domain via a disulfide bond. Particular amino acid residues are believed to form an interface between the light and heavy chain variable domains (Chothia et al., 1985, J. Mol. Biol. 186:651-663.)

Certain domains within the variable domains differ extensively between different antibodies i.e., are “hypervariable.” These hypervariable domains contain residues that are directly involved in the binding and specificity of each particular antibody for its specific antigenic determinant. Hypervariability, both in the light chain and the heavy chain variable domains, is concentrated in three segments known as complementarity determining regions (CDRs) or hypervariable loops (HVLs). CDRs are defined by sequence comparison in Kabat et al., 1991, In: Sequences of Proteins of Immunological Interest, 5^{th} Ed. Public Health Service, National Institutes of Health, Bethesda, Md., whereas HVLs are structurally defined according to the three-dimensional structure of the variable domain, as described by Chothia and Lesk, 1987, J. Mol. Biol. 196: 901-917. Where these two methods result in slightly different identifications of a CDR, the structural definition is preferred. As defined by Kabat, CDR-L1 is positioned at about residues 24-34, CDR-L2, at about residues 50-56, and CDR-L3, at about residues 89-97 in the light chain variable domain; CDR-H1 is positioned at about residues 31-35, CDR-H2 at about residues 50-65, and CDR-H3 at about residues 95-102 in the heavy chain variable domain. The CDR1, CDR2, CDR3 of the heavy and light chains therefore define the unique and functional properties specific for a given antibody.

The three CDRs within each of the heavy and light chains are separated by framework regions (FR), which contain sequences that tend to be less variable. From the amino terminus to the carboxy terminus of the heavy and light chain variable domains, the FRs and CDRs are arranged in the order: FR1, CDR1, FR2, CDR2, FR3, CDR3, and FR4. The largely β-sheet configuration of the FRs brings the CDRs within each of the chains into close proximity to each other as well as to the CDRs from the other chain. The resulting conformation contributes to the antigen binding site (see Kabat et al., 1991, NIH Publ. No. 91-3242, Vol. I, pages 647-669), although not all CDR residues are necessarily directly involved in antigen binding.

FR residues and Ig constant domains are not directly involved in antigen binding, but contribute to antigen binding and/or mediate antibody effector function. Some FR residues are thought to have a significant effect on antigen binding in at least three ways: by noncovalently binding directly to an epitope, by interacting with one or more CDR residues, and by affecting the interface between the heavy and light chains. The constant domains are not directly involved in antigen binding but mediate various Ig effector functions, such as participation of the antibody in antibody-dependent cellular cytotoxicity (ADCC), complement-dependent cytotoxicity (CDC) and antibody-dependent cellular phagocytosis (ADCP).

The light chains of vertebrate immunoglobulins are assigned to one of two clearly distinct classes, kappa (κ) and lambda (λ), based on the amino acid sequence of the constant domain. By comparison, the heavy chains of mammalian immunoglobulins are assigned to one of five major classes, according to the sequence of the constant domains: IgA, IgD, IgE, IgG, and IgM. IgG and IgA are further divided into subclasses (isotypes), e.g., IgG₁, IgG₂, IgG₃, IgG₄, IgA₁, and IgA₂, respectively. The heavy chain constant domains that correspond to the different classes of immunoglobulins are called α, δ, ε, γ, and μ, respectively. The subunit structures and three-dimensional configurations of the classes of native immunoglobulins are well known.

The terms, “antibody”, “anti-Sema3A antibody”, “humanized anti-Sema3A antibody”, and “variant humanized anti-Sema3A antibody” are used herein in the broadest sense and specifically encompass monoclonal antibodies (including full length monoclonal antibodies), multispecific antibodies (e.g., bispecific antibodies), and antibody fragments such as variable domains and other portions of antibodies that exhibit a desired biological activity, e.g., binding to Sema3A.

The term “monoclonal antibody” (mAb) refers to an antibody of a population of substantially homogeneous antibodies; that is, the individual antibodies in that population are identical except for naturally occurring mutations that may be present in minor amounts. Monoclonal antibodies are highly specific, being directed against a single antigenic determinant, an “epitope”. Therefore, the modifier “monoclonal” is indicative of a substantially homogeneous population of antibodies directed to the identical epitope and is not to be construed as requiring production of the antibody by any particular method. It should be understood that monoclonal antibodies can be made by any technique or methodology known in the art; including e.g., the hybridoma method (Kohler et al., 1975, Nature 256:495), or recombinant DNA methods known in the art (see, e.g., U.S. Pat. No. 4,816,567), or methods of isolation of monoclonal recombinantly produced using phage antibody libraries, using techniques described in Clackson et al., 1991, Nature 352: 624-628, and Marks et al., 1991, J. Mol. Biol. 222: 581-597.

Chimeric antibodies consist of the heavy and light chain variable regions of an antibody from one species (e.g., a non-human mammal such as a mouse) and the heavy and light chain constant regions of another species (e.g. human) antibody and can be obtained by linking the DNA sequences encoding the variable regions of the antibody from the first species (e.g., mouse) to the DNA sequences for the constant regions of the antibody from the second (e.g. human) species and transforming a host with an expression vector containing the linked sequences to allow it to produce a chimeric antibody. Alternatively, the chimeric antibody also could be one in which one or more regions or domains of the heavy and/or light chain is identical with, homologous to, or a variant of the corresponding sequence in a monoclonal antibody from another immunoglobulin class or isotype, or from a consensus or germline sequence. Chimeric antibodies can include fragments of such antibodies, provided that the antibody fragment exhibits the desired biological activity of its parent antibody, for example binding to the same epitope (see, e.g., U.S. Pat. No. 4,816,567; and Morrison et al., 1984, Proc. Natl. Acad. Sci. USA 81: 6851-6855).

The terms, “antibody fragment”, “antigen binding fragment”, “anti-Sema3A antibody fragment”, “humanized anti-Sema3A antibody fragment”, “variant humanized anti-Sema3A antibody fragment” refer to a portion of a full length anti-Sema3A antibody, in which a variable region or a functional capability is retained, for example specific Sema3A epitope binding. Examples of antibody fragments include, but are not limited to, a Fab, Fab′, F(ab′)₂, Fd, Fv, scFv and scFv-Fc fragment, a diabody, a linear antibody, a single-chain antibody, a minibody, a diabody formed from antibody fragments, and multispecific antibodies formed from antibody fragments.

Full length antibodies can be treated with enzymes such as papain or pepsin to generate useful antibody fragments. Papain digestion is used to produce two identical antigen-binding antibody fragments called “Fab” fragments, each with a single antigen-binding site, and a residual “Fc” fragment. The Fab fragment also contains the constant domain of the light chain and the C_{H1} domain of the heavy chain. Pepsin treatment yields a F(ab′)₂ fragment that has two antigen-binding sites and is still capable of cross-linking antigen.

Fab′ fragments differ from Fab fragments by the presence of additional residues including one or more cysteines from the antibody hinge region at the C-terminus of the C_{H1} domain. F(ab′)₂ antibody fragments are pairs of Fab′ fragments linked by cysteine residues in the hinge region. Other chemical couplings of antibody fragments are also known.

“Fv” fragment contains a complete antigen-recognition and binding site consisting of a dimer of one heavy and one light chain variable domain in tight, non-covalent association. In this configuration, the three CDRs of each variable domain interact to define an antigen-biding site on the surface of the V_{H}-V_{L} dimer. Collectively, the six CDRs confer antigen-binding specificity to the antibody.

A “single-chain Fv” or “scFv” antibody fragment is a single chain Fv variant comprising the V_{H} and V_{L} domains of an antibody where the domains are present in a single polypeptide chain. The single chain Fv is capable of recognizing and binding antigen. The scFv polypeptide may optionally also contain a polypeptide linker positioned between the V_{H} and V_{L} domains in order to facilitate formation of a desired three-dimensional structure for antigen binding by the scFv (see, e.g., Pluckthun, 1994, In The Pharmacology of monoclonal Antibodies, Vol. 113, Rosenburg and Moore eds., Springer-Verlag, New York, pp. 269-315).

Other recognized antibody fragments include those that comprise a pair of tandem Fd segments (V_{H}-C_{H1}-V_{H}-C_{H1}) to form a pair of antigen binding regions. These “linear antibodies” can be bispecific or monospecific as described in, for example, Zapata et al. 1995, Protein Eng. 8(10):1057-1062.

A humanized antibody or a humanized antibody fragment is a specific type of chimeric antibody which includes an immunoglobulin amino acid sequence variant, or fragment thereof, which is capable of binding to a predetermined antigen and which, comprises one or more FRs having substantially the amino acid sequence of a human immunoglobulin and one or more CDRs having substantially the amino acid sequence of a non-human immunoglobulin. This non-human amino acid sequence often referred to as an “import” sequence is typically taken from an “import” antibody domain, particularly a variable domain. In general, a humanized antibody includes at least the CDRs or HVLs of a non-human antibody, inserted between the FRs of a human heavy or light chain variable domain.

The present invention describes specific humanized anti-Sema3A antibodies which contain CDRs derived from a murine or chimeric antibody inserted between the FRs of human germline sequence heavy and light chain variable domains. It will be understood that certain murine FR residues may be important to the function of the humanized antibodies and therefore certain of the human germline sequence heavy and light chain variable domains residues are modified to be the same as those of the corresponding murine sequence.

As used herein, the expressions “antibody of the invention” and the “anti-Sema3A antibody of the invention” refer to the anti-Sema3A antibody or an antigen-binding fragment thereof described herein. Preferably, said expressions refer to any antibody comprising a heavy chain variable region comprising the amino acid sequence of SEQ ID NO: 1 (H-CDR1); the amino acid sequence of SEQ ID NO: 2 (H-CDR2); and the amino acid sequence of SEQ ID NO: 3 (H-CDR3), and a light chain variable region comprising the amino acid sequence of SEQ ID NO: 4 (L-CDR1); the amino acid sequence of SEQ ID NO: 5 (L-CDR2); and the amino acid sequence of SEQ ID NO: 6 (L-CDR3).

In one aspect, a humanized anti-Sema3A antibody comprises substantially all of at least one, and typically two, variable domains (such as contained, for example, in Fab, Fab′, F(ab′)₂, Fabc, and Fv fragments) in which all, or substantially all, of the CDRs correspond to those of a non-human immunoglobulin, and specifically herein, the CDRs are murine sequences, and the FRs are those of a human immunoglobulin consensus or germline sequence. In another aspect, a humanized anti-Sema3A antibody also includes at least a portion of an immunoglobulin Fc region, typically that of a human immunoglobulin. Ordinarily, the antibody will contain both the light chain as well as at least the variable domain of a heavy chain. The antibody also may include one or more of the C_{H1}, hinge, C_{H2}, C_{H3}, and/or C_{H4} regions of the heavy chain, as appropriate.

A humanized anti-Sema3A antibody can be selected from any class of immunoglobulins, including IgM, IgG, IgD, IgA and IgE, and any isotype, including IgG₁, IgG₂, IgG₃, IgG₄, IgA₁ and IgA₂. For example, the constant domain can be a complement fixing constant domain where it is desired that the humanized antibody exhibit cytotoxic activity, and the isotype is typically IgG₁. Where such cytotoxic activity is not desirable, the constant domain may be of another isotype, e.g., IgG₂. An alternative humanized anti-Sema3A antibody can comprise sequences from more than one immunoglobulin class or isotype, and selecting particular constant domains to optimize desired effector functions is within the ordinary skill in the art. In specific embodiments, the present invention provides antibodies that are IgG₁ antibodies and more particularly IgG₁ antibodies characterized by a reduced effector function.

Preferably, the anti-Sema3A antibody of the invention is a humanized antibody formatted as IgG1KO.

The FRs and CDRs, or HVLs, of a humanized anti-Sema3A antibody do need not to correspond precisely to the parental sequences. For example, one or more residues in the import CDR, or HVL, or the consensus or germline FR sequence may be altered (e.g., mutagenized) by substitution, insertion or deletion such that the resulting amino acid residue is no longer identical to the original residue in the corresponding position in either parental sequence but the antibody nevertheless retains the function of binding to Sema3A. Such alteration typically will not be extensive and will be conservative alterations. Usually, at least 75% of the humanized antibody residues will correspond to those of the parental consensus or germline FR and import CDR sequences, more often at least 90%, and most frequently greater than 95%, or greater than 98% or greater than 99%.

Immunoglobulin residues that affect the interface between heavy and light chain variable regions (“the V_{L}-V_{H} interface”) are those that affect the proximity or orientation of the two chains with respect to one another. Certain residues that may be involved in interchain interactions include V_{L} residues 34, 36, 38, 44, 46, 87, 89, 91, 96, and 98 and V_{H} residues 35, 37, 39, 45, 47, 91, 93, 95, 100, and 103 (utilizing the numbering system set forth in Kabat et al., Sequences of Proteins of Immunological Interest (National Institutes of Health, Bethesda, Md., 1987)). U.S. Pat. No. 6,407,213 also discusses that residues such as V_{L} residues 43 and 85, and V_{H} residues 43 and 60 also may be involved in this interaction. While these residues are indicated for human IgG only, they are applicable across species. Important antibody residues that are reasonably expected to be involved in interchain interactions are selected for substitution into the consensus sequence.

The terms “consensus sequence” and “consensus antibody” refer to an amino acid sequence which comprises the most frequently occurring amino acid residue at each location in all immunoglobulins of any particular class, isotype, or subunit structure, e.g., a human immunoglobulin variable domain. The consensus sequence may be based on immunoglobulins of a particular species or of many species. A “consensus” sequence, structure, or antibody is understood to encompass a consensus human sequence as described in certain embodiments, and to refer to an amino acid sequence which comprises the most frequently occurring amino acid residues at each location in all human immunoglobulins of any particular class, isotype, or subunit structure. Thus, the consensus sequence contains an amino acid sequence having at each position an amino acid that is present in one or more known immunoglobulins, but which may not exactly duplicate the entire amino acid sequence of any single immunoglobulin. The variable region consensus sequence is not obtained from any naturally produced antibody or immunoglobulin. Kabat et al., 1991, Sequences of Proteins of Immunological Interest, 5th Ed. Public Health Service, National Institutes of Health, Bethesda, Md., and variants thereof. The FRs of heavy and light chain consensus sequences, and variants thereof, provide useful sequences for the preparation of humanized anti-Sema3A antibodies. See, for example, U.S. Pat. Nos. 6,037,454 and 6,054,297.

Human germline sequences are found naturally in human population. A combination of those germline genes generates antibody diversity. Germline antibody sequences for the light chain of the antibody come from conserved human germline kappa or lambda v-genes and j-genes. Similarly, the heavy chain sequences come from germline v-, d- and j-genes (LeFranc, M-P, and LeFranc, G, “The Immunoglobulin Facts Book” Academic Press, 2001).

An “isolated” antibody is one that has been identified and separated and/or recovered from a component of its natural environment. Contaminant components of the antibody's natural environment are those materials that may interfere with diagnostic or therapeutic uses of the antibody, and can be enzymes, hormones, or other proteinaceous or nonproteinaceous solutes. In one aspect, the antibody will be purified to at least greater than 95% isolation by weight of antibody.

The term “antibody performance” refers to factors/properties that contribute to antibody recognition of antigen or the effectiveness of an antibody in vivo. In a preferred embodiment, it refers to the ability of the antibody to prevent cytoskeletal collapse in retinal cells. Changes in the amino acid sequence of an antibody can affect antibody properties such as folding, and can influence physical factors such as initial rate of antibody binding to antigen (kₐ), dissociation constant of the antibody from antigen (k_{d}), affinity constant of the antibody for the antigen (Kd), conformation of the antibody, protein stability, and half-life of the antibody.

As used herein, the terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In some embodiments, the two sequences that are compared are the same length after gaps are introduced within the sequences, as appropriate (e.g., excluding additional sequence extending beyond the sequences being compared). For example, when variable region sequences are compared, the leader and/or constant domain sequences are not considered. For sequence comparisons between two sequences, a “corresponding” CDR refers to a CDR in the same location in both sequences (e.g., CDR-H1 of each sequence).

The determination of percent identity or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

As used herein, the expressions “cell”, “cell line”, and “cell culture” are used interchangeably and all such designations include the progeny thereof. Thus, “transformants” and “transformed cells” include the primary subject cell and cultures derived therefrom without regard for the number of transfers.

The term “mammal” for purposes of treatment refers to any animal classified as a mammal, including humans, domesticated and farm animals, and zoo, sports, or pet animals, such as dogs, horses, cats, cows, and the like. Preferably, the mammal is human.

As used herein “thrombotic disease” refers to the formation of a blood clot inside a blood vessel, obstructing the flow of blood through the circulatory system. Preferably, the expression “thrombotic disease of the retina” refers to a thrombotic disease of the retina selected from the group consisting of retinal vein occlusion including central retinal vein occlusion (CRVO), hemispheric retinal vein occlusion (HRVO), branch retinal vein occlusion (BRVO), and arterial occlusive disease of the retina. In a preferred embodiment, the expression “thrombotic disease of the retina” refers to a retinal vein occlusion (RVO).

Retinal vein occlusion is the most common retinal vascular disease after diabetic retinopathy. Depending on the area of retinal venous drainage effectively occluded, it is broadly classified as either central retinal vein occlusion (CRVO), hemispheric retinal vein occlusion (HRVO), or branch retinal vein occlusion (BRVO). It has been observed that each of these has two subtypes. Presentation of RVO in general is with variable painless visual loss with any combination of fundal findings consisting of retinal vascular tortuosity, retinal hemorrhages (blot and flame shaped), cotton wool spots, optic disc swelling and macular edema. In a CRVO, retinal hemorrhages will be found in all four quadrants of the fundus, whilst these are restricted to either the superior or inferior fundal hemisphere in a HRVO. In a BRVO, hemorrhages are largely localized to the area drained by the occluded branch retinal vein. Vision loss occurs secondary to macular edema or ischemia.

A “disease” or “disorder”, as used herein, is any condition that would benefit from treatment with a humanized anti-Sema3A antibody described herein. This includes chronic and acute disorders or diseases including those pathological conditions that predispose the mammal to the disorder in question.

The term “intravitreal injection” has its normal meaning in the art and refers to introduction of an anti-Sema3A antibody or an antigen-binding fragment thereof into the vitreous of a patient.

The term “subcutaneous administration” refers to introduction of an anti-Sema3A antibody or an antigen-binding fragment thereof under the skin of an animal or human patient, preferable within a pocket between the skin and underlying tissue, by relatively slow, sustained delivery from a drug receptacle. Pinching or drawing the skin up and away from underlying tissue may create the pocket.

The term “subcutaneous infusion” refers to introduction of a drug under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human patient, where bolus drug delivery is less than approximately 15 minutes, in another aspect, less than 5 minutes, and in still another aspect, less than 60 seconds. In yet another aspect, administration is within a pocket between the skin and underlying tissue, where the pocket may be created by pinching or drawing the skin up and away from underlying tissue.

The term “therapeutically effective amount” is used to refer to an amount of an anti-Sema3A antibody or an antigen-binding fragment thereof that relieves or ameliorates one or more of the symptoms of the disorders being treated. In doing so it is that amount that has a beneficial patient outcome. Efficacy can be measured in conventional ways, depending on the condition to be treated. For example, in eye/retinal diseases or disorders characterized by cells expressing Sema3A, efficacy can be measured by determining the response rates, e.g. restoration of vision or by assessing the time of delay until disease progression.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an anti-Sema3A antibody or an antigen-binding fragment thereof prior to or following the onset of a symptom of a disease or disorder thereby preventing or removing one or more signs of the disease or disorder. As another example, the term includes the administration of an anti-Sema3A antibody or an antigen-binding fragment thereof after clinical manifestation of the disease to combat the symptoms of the disease. Further, administration of an anti-Sema3A antibody or an antigen-binding fragment thereof after onset and after clinical symptoms have developed where administration affects clinical parameters of the disease or disorder, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the anti-Sema3A antibody composition or an antigen-binding fragment thereof, the result should be considered an effective treatment of the underlying disorder regardless of whether all the symptoms of the disorder are alleviated or not.

The term “package insert” is used to refer to instructions customarily included in commercial packages of therapeutic products, that contain information about the indications, usage, administration, contraindications and/or warnings concerning the use of such therapeutic products.

### Antibody of the Invention for Use for Treating a Thrombotic Disease of the Retina

In a first aspect, the invention relates to an anti-Sema3A antibody or an antigen-binding fragment thereof for use for treating a thrombotic disease of the retina.

In a preferred embodiment, said thrombotic disease of the retina is selected from the group consisting of retinal vein occlusion (RVO) including central retinal vein occlusion (CRVO), hemispheric retinal vein occlusion (HRVO), branch retinal vein occlusion (BRVO), and arterial occlusive disease of the retina.

In another preferred embodiment, said antibody is a humanized anti-Sema3A antibody, more preferably a humanized monoclonal anti-Sema3A antibody.

In an initial characterization, a library of antibodies targeting Sema3A variants was generated by placing the CDRs of murine antibodies into FRs of the human consensus heavy and light chain variable domains and furthermore by engineering the FRs with different alterations. This resulted in a humanized antibody directed against Sema3A with enhanced properties as disclosed herein. The sequences of the antibody of the invention are shown in the table 1 below.

TABLE 1

| | | SEQ ID |
|---|---|---|
| Name | Amino acid sequence | NO |
| HCDR1 | SYYMS | SEQ ID |
| | | NO: 1 |
| HCDR2 | TIIKSGGYAY YPDSVKD | SEQ ID |
| | | NO: 2 |
| HCDR3 | GGQGAMDY | SEQ ID |
| | | NO: 3 |
| LCDR1 | RASQSIGDYL H | SEQ ID |
| | | NO: 4 |
| LCDR2 | YASQSIS | SEQ ID |
| | | NO: 5 |
| LCDR3 | QQGYSFPYT | SEQ ID |
| | | NO: 6 |
| VH- | EVQLVESGGG LVQPGGSLRL SCAASGFTFS | SEQ ID |
| variant | SYYMSWVRQA PGKGLEWVST IIKSGGYAYY | NO: 7 |
| 1 | PDSVKDRFTI SRDNSKNTLY LQMSSLRAED | |
| | TAVYYCVRGG QGAMDYWGQG TTVTVSS | |
| VH- | EVQLVESGGG LVQPGGSLRL SCAASGFPFS | SEQ ID |
| variant | SYYMSWVRQA PGKGLEWVST IIKSGGYAYY | NO: 8 |
| 2 | PDSVKDRFTI SRDNSKNTLY LQMSSLRAED | |
| | TAVYYCVRGG QGAMDYWGQG TTVTVSS | |
| VH- | EVQLVESGGG LVQLGGSLRL SCAASGFTFS | SEQ ID |
| variant | SYYMSWVRQA PGKGLEWVST IIKSGGYAYY | NO: 9 |
| 3 | PDSVKDRFTI SRDNSKNTLY LQMNSLRAED | |
| | TAVYYCVKGG QGAMDYWGQG TTVTVSS | |
| VH- | EVQLVESGGG LLQLGGSLRL SCAASGFTFS | SEQ ID |
| variant | SYYMSWVRQA PGKGLEWVST IIKSGGYAYY | NO: 10 |
| 4 | PDSVKDRFTI SRDNSKNTLN LQMNSLRAED | |
| | TAVYYCVKGG QGAMDYWGQG TTVTVSS | |
| VL- | EIVLTQSPAT LSLSPGERAT LSCRASQSIG | SEQ ID |
| variant | DYLHWYQQKP GQAPRLLIKY ASQSISGIPA | NO: 11 |
| a | RFSGSGSGTD FTLTITSLEP EDFAVYYCQQ | |
| | GYSFPYTFGG GTKLEIK | |
| VL- | EIVLTQSPAT LSLSPGERAT LSCRASQSIG | SEQ ID |
| variant | DYLHWYQQKP GQAPRLLIYY ASQSISGIPA | NO: 12 |
| b | RFSGSGSGTD FTLTISSLEP EDFAVYYCQQ | |
| | GYSFPYTFGG GTKLEIK | |
| VL- | EIVLTQSPAT LSLSPGERAT LSCRASQSIG | SEQ ID |
| variant | DYLHWYQQKP GQAPRLLIKY ASQSISGIPA | NO: 13 |
| c | RFSGSGSGTD FTLTISSLEP EDFAVYYCQQ | |
| | GYSFPYTFGG GTKLEIK | |
| Heavy | EVQLVESGGG LVQPGGSLRL SCAASGFTFS | SEQ ID |
| Chain- | SYYMSWVRQA PGKGLEWVST IIKSGGYAYY | NO: 14 |
| Clone | PDSVKDRFTI SRDNSKNTLY LQMSSLRAED | |
| I | TAVYYCVRGG QGAMDYWGQG TTVTVSSAST | |
| | KGPSVFPLAP SSKSTSGGTA ALGCLVKDYF | |
| | PEPVTVSWNS GALTSGVHTF PAVLQSSGLY | |
| | SLSSVVTVPS SSLGTQTYIC NVNHKPSNTK | |
| | VDKRVEPKSC DKTHTCPPCP APEAAGGPSV | |
| | FLFPPKPKDT LMISRTPEVT CVVVDVSHED | |
| | PEVKFNWYVD GVEVHNAKTK PREEQYNSTY | |
| | RVVSVLTVLH QDWLNGKEYK CKVSNKALPA | |
| | PIEKTISKAK GQPREPQVYT LPPSREEMTK | |
| | NQVSLTCLVK GFYPSDIAVE WESNGQPENN | |
| | YKTTPPVLDS DGSFFLYSKL TVDKSRWQQG | |
| | NVFSCSVMHE ALHNHYTQKS LSLSPG | |
| Light | EIVLTQSPAT LSLSPGERAT LSCRASQSIG | SEQ ID |
| Chain- | DYLHWYQQKP GQAPRLLIKY ASQSISGIPA | NO: 15 |
| Clone | RFSGSGSGTD FTLTITSLEP EDFAVYYCQQ | |
| I | GYSFPYTFGG GTKLEIKRTV AAPSVFIFPP | |
| | SDEQLKSGTA SVVCLLNNFY PREAKVQWKV | |
| | DNALQSGNSQ ESVTEQDSKD STYSLSSTLT | |
| | LSKADYEKHK VYACEVTHQG LSSPVTKSFN | |
| | RGEC | |
| Heavy | EVQLVESGGG LVQPGGSLRL SCAASGFPFS | SEQ ID |
| Chain- | SYYMSWVRQA PGKGLEWVST IIKSGGYAYY | NO: 16 |
| Clone | PDSVKDRFTI SRDNSKNTLY LQMSSLRAED | |
| II | TAVYYCVRGG QGAMDYWGQG TTVTVSSAST | |
| | KGPSVFPLAP SSKSTSGGTA ALGCLVKDYF | |
| | PEPVTVSWNS GALTSGVHTF PAVLQSSGLY | |
| | SLSSVVTVPS SSLGTQTYIC NVNHKPSNTK | |
| | VDKRVEPKSC DKTHTCPPCP APEAAGGPSV | |
| | FLFPPKPKDT LMISRTPEVT CVVVDVSHED | |
| | PEVKFNWYVD GVEVHNAKTK PREEQYNSTY | |
| | RVVSVLTVLH QDWLNGKEYK CKVSNKALPA | |
| | PIEKTISKAK GQPREPQVYT LPPSREEMTK | |
| | NQVSLTCLVK GFYPSDIAVE WESNGQPENN | |
| | YKTTPPVLDS DGSFFLYSKL TVDKSRWQQG | |
| | NVFSCSVMHE ALHNHYTQKS LSLSPG | |
| Heavy | EVQLVESGGG LVQLGGSLRL SCAASGFTFS | SEQ ID |
| Chain- | SYYMSWVRQA PGKGLEWVST IIKSGGYAYY | NO: 17 |
| Clone | PDSVKDRFTI SRDNSKNTLY LQMNSLRAED | |
| III | TAVYYCVKGG QGAMDYWGQG TTVTVSSAST | |
| | KGPSVFPLAP SSKSTSGGTA ALGCLVKDYF | |
| | PEPVTVSWNS GALTSGVHTF PAVLQSSGLY | |
| | SLSSVVTVPS SSLGTQTYIC NVNHKPSNTK | |
| | VDKRVEPKSC DKTHTCPPCP APEAAGGPSV | |
| | FLFPPKPKDT LMISRTPEVT CVVVDVSHED | |
| | PEVKFNWYVD GVEVHNAKTK PREEQYNSTY | |
| | RVVSVLTVLH QDWLNGKEYK CKVSNKALPA | |
| | PIEKTISKAK GQPREPQVYT LPPSREEMTK | |
| | NQVSLTCLVK GFYPSDIAVE WESNGQPENN | |
| | YKTTPPVLDS DGSFFLYSKL TVDKSRWQQG | |
| | NVFSCSVMHE ALHNHYTQKS LSLSPG | |
| Light | EIVLTQSPAT LSLSPGERAT LSCRASQSIG | SEQ ID |
| Chain- | DYLHWYQQKP GQAPRLLIYY ASQSISGIPA | NO: 18 |
| Clone | RFSGSGSGTD FTLTISSLEP EDFAVYYCQQ | |
| III | GYSFPYTFGG GTKLEIKRTV AAPSVFIFPP | |
| | SDEQLKSGTA SVVCLLNNFY PREAKVQWKV | |
| | DNALQSGNSQ ESVTEQDSKD STYSLSSTLT | |
| | LSKADYEKHK VYACEVTHQG LSSPVTKSFN | |
| | RGEC | |
| Heavy | EVQLVESGGG LLQLGGSLRL SCAASGFTFS | SEQ ID |
| Chain- | SYYMSWVRQA PGKGLEWVST IIKSGGYAYY | NO: 19 |
| Clone | PDSVKDRFTI SRDNSKNTLN LQMNSLRAED | |
| IV | TAVYYCVKGG QGAMDYWGQG TTVTVSSAST | |
| | KGPSVFPLAP SSKSTSGGTA ALGCLVKDYF | |
| | PEPVTVSWNS GALTSGVHTF PAVLQSSGLY | |
| | SLSSVVTVPS SSLGTQTYIC NVNHKPSNTK | |
| | VDKRVEPKSC DKTHTCPPCP APEAAGGPSV | |
| | FLFPPKPKDT LMISRTPEVT CVVVDVSHED | |
| | PEVKFNWYVD GVEVHNAKTK PREEQYNSTY | |
| | RVVSVLTVLH QDWLNGKEYK CKVSNKALPA | |
| | PIEKTISKAK GQPREPQVYT LPPSREEMTK | |
| | NQVSLTCLVK GFYPSDIAVE WESNGQPENN | |
| | YKTTPPVLDS DGSFFLYSKL TVDKSRWQQG | |
| | NVFSCSVMHE ALHNHYTQKS LSLSPG | |
| Light | EIVLTQSPAT LSLSPGERAT LSCRASQSIG | SEQ ID |
| Chain- | DYLHWYQQKP GQAPRLLIKY ASQSISGIPA | NO: 20 |
| Clone | RFSGSGSGTD FTLTISSLEP EDFAVYYCQQ | |
| IV | GYSFPYTFGG GTKLEIKRTV AAPSVFIFPP | |
| | SDEQLKSGTA SVVCLLNNFY PREAKVQWKV | |
| | DNALQSGNSQ ESVTEQDSKD STYSLSSTLT | |
| | LSKADYEKHK VYACEVTHQG LSSPVTKSFN | |
| | RGEC | |

In one embodiment, the present invention provides an anti-Sema3A antibody or an antigen-binding fragment thereof for use for treating a thrombotic disease of the retina, wherein said anti-Sema3A antibody or antigen-binding fragment thereof comprises:
a heavy chain variable region comprising the amino acid sequence of SEQ ID NO: 1 (H-CDR1); the amino acid sequence of SEQ ID NO: 2 (H-CDR2); and the amino acid sequence of SEQ ID NO: 3 (H-CDR3); and
a light chain variable region comprising the amino acid sequence of SEQ ID NO: 4 (L-CDR1); the amino acid sequence of SEQ ID NO: 5 (L-CDR2); and the amino acid sequence of SEQ ID NO: 6 (L-CDR3).

In another embodiment, the present invention provides an anti-Sema3A antibody or an antigen-binding fragment thereof for use for treating a thrombotic disease of the retina, wherein said anti-Sema3A antibody or antigen-binding fragment thereof comprises:
a heavy chain variable region comprising an amino acid sequence at least 80%, at least 90%, at least 95%, at least 98%, or at least 99% identical to the amino acid sequence SEQ ID NO: 7, SEQ ID NO: 8, SEQ ID NO: 9 or SEQ ID NO: 10; and
a light chain variable region comprising an amino acid sequence at least 80%, at least 90%, at least 95%, at least 98%, or at least 99% identical to the amino acid sequence of SEQ ID NO: 11, SEQ ID NO: 12 or SEQ ID NO: 13.

In another embodiment, the present invention provides an anti-Sema3A antibody or an antigen-binding fragment thereof for use for treating a thrombotic disease of the retina, wherein said anti-Sema3A antibody or antigen-binding fragment thereof comprises:
a heavy chain variable region comprising an amino acid sequence at least 80%, at least 90%, at least 95%, at least 98%, or at least 99% identical to the amino acid sequence SEQ ID NO: 7, SEQ ID NO: 8, SEQ ID NO: 9 or SEQ ID NO: 10; and
a light chain variable region comprising an amino acid sequence at least 80%, at least 90%, at least 95%, at least 98%, or at least 99% identical to the amino acid sequence of SEQ ID NO: 11, SEQ ID NO: 12 or SEQ ID NO: 13;
   wherein:
the heavy chain variable region comprises the amino acid sequence of SEQ ID NO: 1 (H-CDR1); the amino acid sequence of SEQ ID NO: 2 (H-CDR2); and the amino acid sequence of SEQ ID NO: 3 (H-CDR3); and
the light chain variable region comprises the amino acid sequence of SEQ ID NO: 4 (L-CDR1); the amino acid sequence of SEQ ID NO: 5 (L-CDR2); and the amino acid sequence of SEQ ID NO: 6 (L-CDR3).

In yet another embodiment, the present invention provides an anti-Sema3A antibody or an antigen-binding fragment thereof for use for treating a thrombotic disease of the retina, wherein said anti-Sema3A antibody or antigen-binding fragment thereof comprises:
a heavy chain variable region comprising the amino acid sequences of SEQ ID NO: 7, SEQ ID NO: 8, SEQ ID NO: 9 or SEQ ID NO: 10; and
a light chain variable region comprising the amino acid sequence of SEQ ID NO: 11, SEQ ID NO: 12 or SEQ ID NO: 13.

In a preferred embodiment, the invention provides an anti-Sema3A antibody or an antigen-binding fragment thereof for use for treating a thrombotic disease of the retina, wherein said anti-Sema3A antibody or antigen-binding fragment thereof comprises:
a variable heavy chain and a variable light chain comprising the amino acid sequences of SEQ ID NO: 7 and SEQ ID NO: 11, respectively;
a variable heavy chain and a variable light chain comprising the amino acid sequences of SEQ ID NO: 8 and SEQ ID NO: 11, respectively;
a variable heavy chain and a variable light chain comprising the amino acid sequences of SEQ ID NO: 9 and SEQ ID NO: 12, respectively; or
a variable heavy chain and a variable light chain comprising the amino acid sequences of SEQ ID NO: 10 and SEQ ID NO: 13, respectively.

In yet another embodiment, the present invention provides an anti-Sema3A antibody or an antigen-binding fragment thereof for use for treating a thrombotic disease of the retina, wherein said anti-Sema3A antibody or antigen-binding fragment thereof comprises:
a heavy chain comprising, preferably consisting of, the amino acid sequence of SEQ ID NO: 14, SEQ ID NO: 16, SEQ ID NO: 17 or SEQ ID NO: 19; and
a light chain comprising, preferably consisting of, the amino acid sequence of SEQ ID NO: 15, SEQ ID NO: 18 or SEQ ID NO: 20.

In a particular embodiment, the invention relates to an anti-Sema3A antibody or an antigen-binding fragment thereof for use for treating a thrombotic disease of the retina, wherein said anti-Sema3A antibody or antigen-binding fragment thereof comprises:
a. a heavy chain comprising the amino acid sequence of SEQ ID NO: 14 and a light chain comprising the amino acid sequence of SEQ ID NO: 15, said antibody being referred to as “clone l”;
b. a heavy chain comprising the amino acid sequence of SEQ ID NO: 16 and a light chain comprising the amino acid sequence of SEQ ID NO: 15, said antibody being referred to as “clone II”;
c. a heavy chain comprising the amino acid sequence of SEQ ID NO: 17 and a light chain comprising the amino acid sequence of SEQ ID NO: 18, said antibody being referred to as “clone III”; or
d. a heavy chain comprising the amino acid sequence of SEQ ID NO: 19 and a light chain comprising the amino acid sequence of SEQ ID NO: 20, said antibody being referred to as “clone IV”.

IgG1-KO mutants have been made by introducing mutations in the Fc region. Mutations to reduce or inhibit effector function are well known by the skilled person and thoroughly disclosed in prior art, for example in Wang et al, Protein Cell 2018, 9(1):63-73 and Stewart et al. Journal for Immuno Therapy of Cancer 2014, 2:29. Typically, a non-limiting list of mutations introduced in the IgG1 Fc region in order to reduce the effector function of the Fc comprises:
L234A and L235A;
L234A, L235A, and N297Q;
L234A, L235A, and P329G; or
L234A, L235A, and D265A;
   wherein the residues are numbered according to the EU index of Kabat.

In a preferred embodiment, the antibody of the invention comprises the two mutations L234A and L235A in the Fc region to reduce effector function.

The CDR disclosed herein and depicted in SEQ ID NO: 1 to 6 are presented according to the Kabat numbering and are summarized in table 2 below with the Kabat position.

TABLE 2

| | | | SEQ |
|---|---|---|---|
| | | Kabat | ID |
| CDR | Kabat Sequence | position | NO: |
| HCDR1 | SYYMS | 31-35 | 1 |
| HCDR2 | TIIKSGGYAYYPDSVKD | 50-66 | 2 |
| HCDR3 | GGQGAMDY | 99-106 | 3 |
| LCDR1 | RASQSIGDYLH | 24-34 | 4 |
| LCDR2 | YASQIS | 50-56 | 5 |
| LCDR3 | QQGYSFPYT | 89-97 | 6 |

The anti-Sema3A antibody of the present invention binds with high affinity to human Sema3A. In an embodiment relating to this aspect, an anti-Sema3A antibody of the present invention binds to human Sema3A at a K_{D}<50 μM. In another embodiment, the anti-Sema3A antibody of the present invention binds to human Sema3A at a K_{D}<35 μM, as exemplified in Example 2. In a preferred embodiment, the anti-Sema3A antibody of the present invention binds to human Sema3A at a K_{D}<30 μM.

The anti-Sema3A antibody of the invention also binds to cyno-Sema3A, mouse Sema3A, rat Sema3A and rabbit Sema3A.

The anti-Sema3A antibody of the present invention prevents Sema3A-induced cytoskeletal collapse in retinal cells with a functional potency of less than 100 μM, preferably less than 80 μM, more preferably less than 70 μM. In a preferred embodiment, the anti-Sema3A antibody of the present invention prevents Sema3A-induced cytoskeletal collapse in retinal cells with a functional potency of 69 μM, as exemplified in Example 2.

In a further aspect, the anti-Sema3A antibody of the present invention proved to have a low immunogenicity risk as described in Example 3. This relies on an in silico prediction of the immunogenicity of the antibody. The immunogenicity risk is typically assessed by various methods well known such as by computer algorithm for predicting T cell epitopes, a major immunogenicity-influencing factor.

It has been indeed reported that sequences containing T-cell epitopes present in proteins of interest could be predicted by using an algorithm based on a computational matrix approach, available under the name EpiMatrix (produced by EpiVax). The person skilled in the art may refer to Van Walle et al., Expert Opin Biol Ther. 2007 March; 7(3): 405-18 and Jawa and al., Clin Immunol. 2013 December; 149(3):534-55.

The inventors have shown that the antibody of the invention shows more advantageous properties than other antibodies or fragments targeting Sema3A mentioned in prior art and described herein.

The inventors have compared the binding affinity of an antibody targeting Sema3A disclosed in WO2014123186 (Chiome Bioscience) with the affinity of the antibody of the present invention. The antibodies of WO2014123186 are disclosed for use in the treatment of Alzheimer's disease. The present Example 4 shows that the antibody of the invention proved to have higher binding affinities for human Sema3A than the prior art antibody disclosed by Chiome Bioscience.

The inventors have also compared the properties of the antibody in accordance with the present invention with the ScFv fragments as disclosed in WO2017074013 (Samsung). These fragments are disclosed for use in treatment of various cancers. The present Example 5 shows that the antibody of the invention proved to have higher binding affinities for human Sema3A than the prior art antibody fragments disclosed by WO2017074013.

A higher binding affinity prolongs the time for neutralization of Sema3A after intravitreal injection of the antibody and allows a reduced injection frequency. A higher binding affinity further allows the administration of a lower dose, limiting the potential side effects. The antibody of the invention thus provides technical advantages over the prior art antibodies. The improved binding affinity and reduced injection frequency considerably ameliorate the efficacy of the treatment of patients in need thereof. It also provides valuable benefits for the patient, especially an improved drug observance and compliance.

### Humanization and Amino Acid Sequence Variants

Further variant anti-Sema3A antibodies and antibody fragments can be engineered based on the set of CDRs identified under the sequences depicted in SEQ ID NO: 1 to 6. It is to be understood that in said variant anti-Sema3A antibodies and antibody fragments the amino acid sequence of the CDRs remain unchanged but the surrounding regions e.g. FR regions can be engineered. Amino acid sequence variants of the anti-Sema3A antibody can be prepared by introducing appropriate nucleotide changes into the anti-Sema3A antibody DNA, or by peptide synthesis. Such variants include, for example, deletions from, and/or insertions into and/or substitutions of, residues within the amino acid sequences of the anti-Sema3A antibodies of the examples herein. Any combination of deletions, insertions, and substitutions is made to arrive at the final construct, provided that the final construct possesses the desired characteristics. The amino acid changes also may alter post-translational processes of the humanized or variant anti-Sema3A antibody, such as changing the number or position of glycosylation sites.

Another type of amino acid variant of the antibody involves altering the original glycosylation pattern of the antibody. The term “altering” in this context means deleting one or more carbohydrate moieties found in the antibody, and/or adding one or more glycosylation sites that were not previously present in the antibody.

In one aspect, the present invention includes nucleic acid molecules that encode the amino acid sequence variants of the anti-Sema3A antibodies described herein. Nucleic acid molecules encoding amino acid sequence variants of the anti-Sema3A antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-Sema3A antibody.

In certain embodiments, the anti-Sema3A antibody is an antibody fragment. There are techniques that have been developed for the production of antibody fragments. Fragments can be derived via proteolytic digestion of intact antibodies (see, e.g., Morimoto et al., 1992, Journal of Biochemical and Biophysical Methods 24:107-117; and Brennan et al., 1985, Science 229:81). Alternatively, the fragments can be produced directly in recombinant host cells. For example, Fab′-SH fragments can be directly recovered from E. coli and chemically coupled to form F(ab′)2 fragments (see, e.g., Carter et al., 1992, Bio/Technology 10:163-167). By another approach, F(ab′)2 fragments can be isolated directly from recombinant host cell culture. Other techniques for the production of antibody fragments will be apparent to the skilled practitioner.

The anti-Sema3A antibodies and antigen-binding fragments thereof can include modifications.

In certain embodiments, it may be desirable to use an anti-Sema3A antibody fragment, rather than an intact antibody. It may be desirable to modify the antibody fragment in order to increase its serum half-life. This can be achieved, for example, by incorporation of a salvage receptor binding epitope into the antibody fragment. In one method, the appropriate region of the antibody fragment can be altered (e.g., mutated), or the epitope can be incorporated into a peptide tag that is then fused to the antibody fragment at either end or in the middle, for example, by DNA or peptide synthesis. See, e.g., WO 96/32478.

In other embodiments, the present invention includes covalent modifications of the anti-Sema3A antibodies. Covalent modifications include modification of cysteinyl residues, histidyl residues, lysinyl and amino-terminal residues, arginyl residues, tyrosyl residues, carboxyl side groups (aspartyl or glutamyl), glutaminyl and asparaginyl residues, or seryl, or threonyl residues. Another type of covalent modification involves chemically or enzymatically coupling glycosides to the antibody. Such modifications may be made by chemical synthesis or by enzymatic or chemical cleavage of the antibody, if applicable. Other types of covalent modifications of the antibody can be introduced into the molecule by reacting targeted amino acid residues of the antibody with an organic derivatizing agent that is capable of reacting with selected side chains or the amino- or carboxy-terminal residues.

Removal of any carbohydrate moieties present on the antibody can be accomplished chemically or enzymatically. Chemical deglycosylation is described by Hakimuddin et al., 1987, Arch. Biochem. Biophys. 259:52 and by Edge et al., 1981, Anal. Biochem., 118:131. Enzymatic cleavage of carbohydrate moieties on antibodies can be achieved by the use of a variety of endo- and exo-glycosidases as described by Thotakura et al., 1987, Meth. Enzymol 138:350.

Another type of useful covalent modification comprises linking the antibody to one of a variety of nonproteinaceous polymers, e.g., polyethylene glycol, polypropylene glycol, or polyoxyalkylenes, in the manner set forth in one or more of U.S. Pat. Nos. 4,640,835, 4,496,689, 4,301,144, 4,670,417, 4,791,192 and 4,179,337.

### Epitope Binding

In a second aspect, the invention relates to an antibody that recognizes a specific “Sema3A antigen epitope” and “Sema3A epitope” for use for treating a thrombotic disease of the retina. In particular, said antibody or fragment thereof binds to an epitope of the human Sema3A with the SEQ ID NO: 22.

In one aspect, the invention relates to an anti-Sema3A antibody or an antigen-binding fragment thereof for use for treating a thrombotic disease of the retina, wherein said anti-Sema3A antibody or antigen-binding fragment thereof binds to at least one amino acid residue within amino acid regions 370-382 of human Sema3A as set forth in SEQ ID NO: 22.

In another aspect, the invention relates to an anti-Sema3A antibody or an antigen-binding fragment thereof for use for treating a thrombotic disease of the retina, wherein said anti-Sema3A antibody or antigen-binding fragment thereof binds to SEQ ID NO: 21.

The sequences SEQ ID NO: 21 and 22 are depicted in the table 3 below.

TABLE 3

| | | SEQ |
|---|---|---|
| | | ID |
| Name | Sequence | NO: |
| Sema3A | DSTKDLPDDV ITF | 21 |
| epitope | | |
| Human | NYQNGKNNVPRLKLSYKEMLESNNVITFNGL | 22 |
| Sema3A | ANSSSYHTFLLDEERSRLYVGAKDHIFSFDL | |
| | VNIKDFQKIVWPVSYTRRDECKWAGKDILKE | |
| | CANFIKVLKAYNQTHLYACGTGAFHPICTYI | |
| | EIGHHPEDNIFKLENSHFENGRGKSPYDPKL | |
| | LTASLLIDGELYSGTAADFMGRDFAIFRTLG | |
| | HHHPIRTEQHDSRWLNDPKFISAHLISESDN | |
| | PEDDKVYFFFRENAIDGEHSGKATHARIGQI | |
| | CKNDFGGHRSLVNKWTTFLKARLICSVPGPN | |
| | GIDTHFDELQDVFLMNFKDPKNPVVYGVFTT | |
| | SSNIFKGSAVCMYSMSDVRRVFLGPYAHRDG | |
| | PNYQWVPYQGRVPYPRPGTCPSKTFGGFDST | |
| | KDLPDDVITFARSHPAMYNPVFPMNNRPIVI | |
| | KTDVNYQFTQIVVDRVDAEDGQYDVMFIGTD | |
| | VGTVLKVVSIPKETWYDLEEVLLEEMTVFRE | |
| | PTAISAMELSTKQQQLYIGSTAGVAQLPLHR | |
| | CDIYGKACAECCLARDPYCAWDGSACSRYFP | |
| | TAKRRTRRQDIRNGDPLTHCSDLHHDNHHGH | |
| | SPEERIIYGVENSSTFLECSPKSQRALVYWQ | |
| | FQRRNEERKEEIRVDDHIIRTDQGLLLRSLQ | |
| | QKDSGNYLCHAVEHGFIQTLLKVTLEVIDTE | |
| | HLEELLHKDDDGDGSKTKEMSNSMTPSQKVW | |
| | YRDFMQLINHPNLNTMDEFCEQVWKRDRKQR | |
| | RQRPGHTPGNSNKWKHLQENKKGRNRRTHEF | |
| | ERAPRSV | |

As used herein, the terms “Sema3A antigen epitope” and “Sema3A epitope” refer to a molecule (e.g., a peptide) or a fragment of a molecule capable of binding to an anti-Sema3A antibody or an antigen-binding fragment thereof. These terms further include, for example, a Sema3A antigenic determinant recognized by any of the antibodies or antibody fragments of the present invention, which has a light and heavy chain CDR combination selected from heavy chain CDRs of the SEQ ID NOs 1 to 3 and light chain CDRs of the SEQ ID NOs: 4 to 6.

Sema3A antigen epitopes can be included in proteins, protein fragments, peptides or the like. The epitopes are most commonly proteins, short oligopeptides, oligopeptide mimics (i.e., organic compounds that mimic antibody binding properties of the Sema3A antigen), or combinations thereof.

It has been found that the antibodies or antibody fragments of the present invention bind to a unique epitope of the human Sema3A. Preferably, an anti-Sema3A antibody or an antigen-binding fragment thereof binds to at least one amino acid residue within amino acid regions 370-382 of the extracellular domain of human Sema3A with the SEQ ID NO: 22. This epitope is located close to the interface of Sema3A and a Plexin A receptor. Binding of the antibody to this epitope inhibits the formation of the signaling holoreceptor complex of the ligand Sema3A, the receptor Plexin A and the co-receptor Nrp1, leading to the interference with the biological effects of such signaling.

In the context of epitope binding, the phrase “binds within amino acid regions X-Y . . . ” means that the anti-Sema3A antibody or an antigen-binding fragment thereof binds to at least one, preferably all of the, amino acid residue within the amino acid region specified in the sequence.

In another aspect, an anti-Sema3A antibody or an antigen-binding fragment thereof binds to at least 10%, 20%, 30%, 40%, 50%, 60%, 70%, 80%, 85%, 90%, 95%, or 100% of the amino acid sequence depicted in SEQ ID NO: 22. Preferably, an anti-Sema3A antibody or an antigen-binding fragment thereof binds to SEQ ID NO: 22.

### Therapeutic Uses

In one embodiment, the present invention provides an anti-Sema3A or an antigen-binding fragment for use for treating a thrombotic disease of the retina by inhibiting the vasorepressive effect of SemaA, by improving revascularisation of the retina, and/or by reducing permeability of blood retinal barrier. The inventors have indeed developed an antibody targeting Sema3A, which is extremely helpful for:
redirecting angiogenesis towards ischemic regions, in order to improve revascularisation of the retina;
preventing pathological neovascularization of the vitreous region; and
preventing blood retinal barrier breakdown.

As previously mentioned, Sema3A is a vasorepulsive cue secreted by hypoxic retinal ganglion cells. By binding to neuropilin-1, it activates the intracellular signalling of plexin receptors on endothelial cells resulting in disassembly of actin fibers. This leads to a cytoskeletal collapse in the filopodia of tip cells, specialized endothelial cells which are directing the growth of new vessels and prevents vascular regeneration of ischemic areas in the retina. The inventors have shown that modulating the vasorepulsive action with a neutralizing Sema3A-antibody would increase the number of tip cells and redirect angiogenesis towards ischemic regions, such as the pathologically enlarged foveal avascular zone in humans with diabetic macular ischemia.

The inventors have shown in Example 1 the relevance and superiority of the therapeutic strategy based on the use of the anti-Sema3A antibody of the invention. They have indeed shown that the antibody of the invention ameliorates cystoid edema and suppresses retinal thinning in inner nuclear layer of RVO murine model. The inventors further have shown that ocular blood flow is improved by the administration of anti-Sema3A antibody of the invention in RVO murine model. Finally, the inventors have exemplified that the anti-Sema3A antibody of the invention reduces the size of retinal non-perfused areas in RVO murine model.

In one embodiment, the present invention provides an anti-Sema3A or an antigen-binding fragment for use for treating a thrombotic disease of the retina, by inhibiting the vasorepressive effect of SemaA, improving revascularisation of the retina, and/or by reducing permeability of blood retinal barrier.

In a preferred embodiment, the present invention provides an anti-Sema3A antibody or an antigen-binding fragment thereof for use for treating a thrombotic disease of the retina in a patient suffering from diabetic macular ischemia, preferably by promoting vascular regeneration the ischemic retina (revascularization) and preventing pathological neovascularization of the vitreous region of the eye.

In another preferred embodiment, the present invention provides an anti-Sema3A antibody or an antigen-binding fragment thereof for use for treating a thrombotic disease of the retina in a patient suffering from diabetic macular edema, preferably by reducing permeability of blood retinal barrier.

In another preferred embodiment, the present invention provides an anti-Sema3A antibody or an antigen-binding fragment thereof for use for treating a thrombotic disease of the retina, by inhibiting Sema3A-induced permeability of the blood retinal barrier and/or Sema3A-induced vasoregression from ischemic areas.

In a fourth aspect, the present invention provides a pharmaceutical composition comprising an anti-Sema3A antibody or an antigen-binding fragment thereof and a pharmaceutically acceptable carrier for use for treating a thrombotic disease of the retina.

The anti-Sema3A antibody or an antigen-binding fragment thereof or the pharmaceutical composition of the invention is administered by any suitable means, including intravitreal, oral, parenteral, subcutaneous, intraperitoneal, intrapulmonary, and intranasal. Parenteral infusions include intramuscular, intravenous, intraarterial, intraperitoneal, or subcutaneous administration. In addition, the anti-Sema3A antibody is suitably administered by pulse infusion, particularly with declining doses of the antibody. In one aspect, the dosing is given by injections, most preferably intravenous or subcutaneous injections, depending in part on whether the administration is brief or chronic. Preferably, the anti-Sema3A antibody is given through an intravitreal injection into the eye.

For the prevention or treatment of disease, the appropriate dosage of antibody will depend on a variety of factors such as the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

In a preferred embodiment, the dose range of the antibodies of the invention applicable per injection is usually from 1 mg/eye to 10 mg/eye, preferably between 1.5 mg/eye and 5 mg/eyes, more preferably between 2 mg/eye and 3 mg/eye and even more preferably about 2.5 mg/eye.

The term “suppression” is used herein in the same context as “amelioration” and “alleviation” to mean a lessening or diminishing of one or more characteristics of the disease.

The antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations, and is the minimum amount necessary to prevent, ameliorate, or treat the eye or retinal diseases addressed by the antibody of the invention.

The antibody need not be, but is optionally, formulated with one or more agents currently used to prevent or treat thrombotic diseases of the retina. The effective amount of such other agents depends on the amount of anti-Sema3A antibody present in the formulation, the type of disorder or treatment, and other factors discussed above. These are generally used in the same dosages and with administration routes as used hereinbefore or about from 1 to 99% of the heretofore employed dosages.

Various delivery systems are known and can be used to administer the anti-Sema3A antibody or an antigen-binding fragment thereof. Methods of introduction include but are not limited to intravitreal, eye drops, intradermal, intramuscular, intraperitoneal, intravenous, subcutaneous, intranasal, epidural, and oral routes. The anti-Sema3A antibody or an antigen-binding fragment thereof can be administered, for example by infusion, bolus or injection, and can be administered together with other biologically active agents. Administration can be systemic or local. In preferred embodiments, the administration is by intravitreal injection. Formulations for such injections may be prepared in, for example, prefilled syringes.

An anti-Sema3A antibody or an antigen-binding fragment thereof can be administered as pharmaceutical compositions comprising a therapeutically effective amount of the anti-Sema3A antibody or an antigen-binding fragment thereof and one or more pharmaceutically compatible ingredients.

In typical embodiments, the pharmaceutical composition is formulated in accordance with routine procedures as a pharmaceutical composition adapted for intravenous or subcutaneous administration to human beings. Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

Further, the pharmaceutical composition can be provided as a pharmaceutical kit comprising (a) a container containing an anti-Sema3A antibody or an antigen-binding fragment thereof in lyophilized form and (b) a second container containing a pharmaceutically acceptable diluent (e.g., sterile water) for injection. The pharmaceutically acceptable diluent can be used for reconstitution or dilution of the lyophilized anti-Sema3A antibody or an antigen-binding fragment thereof. Optionally associated with such container(s) can be a notice in the form prescribed by a governmental agency regulating the manufacture, use or sale of pharmaceuticals or biological products, which notice reflects approval by the agency of manufacture, use or sale for human administration.

The amount of the anti-Sema3A antibody or an antigen-binding fragment thereof that is effective in the treatment or prevention of an eye or retinal disease can be determined by standard clinical techniques. In addition, in vitro assays may optionally be employed to help identify optimal dosage ranges. The precise dose to be employed in the formulation will also depend on the route of administration, and the stage of disorder, and should be decided according to the judgment of the practitioner and each patient's circumstances. Effective doses may be extrapolated from dose-response curves derived from in vitro or animal model test systems.

For example, toxicity and therapeutic efficacy of the anti-Sema3A antibody or an antigen-binding fragment thereof can be determined in cell cultures or experimental animals by standard pharmaceutical procedures for determining the ED₅₀ (the dose therapeutically effective in 50% of the population). An anti-Sema3A antibody or an antigen-binding fragment thereof that exhibits a large therapeutic index is preferred.

The data obtained from the cell culture assays and animal studies can be used in formulating a range of dosage for use in humans. The dosage of the anti-Sema3A antibody or an antigen-binding fragment thereof typically lies within a range of circulating concentrations that include the ED₅₀ with little or no toxicity. The dosage may vary within this range depending upon the dosage form employed and the route of administration utilized. For any anti-Sema3A antibody or an antigen-binding fragment thereof used in the method, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography, ELISA and the like.

For intravitreal injection of the anti-Sema3A antibody, generally longer intervals between treatments are preferred. Due to its improved binding affinity and potency, the anti-Sema3A antibodies of the present invention can be administered in longer intervals.

In one embodiment the anti-Sema3A antibody is administered every 6 weeks, preferably every 7 weeks, preferably every 8 weeks, preferably every 9 weeks, preferably every 10 weeks, preferably every 11 weeks, and more preferably every 12 weeks. In a yet preferred embodiment, the anti-Sema3A antibody of the invention is administered once every 3 months.

Since the volume that can be administered to the eye is strictly limited, it is very important that an anti-Sema3A antibody can be formulated to high concentrations. Furthermore, potency of the anti-Sema3A antibody is of great importance as a potent antibody can exert its effect at even lower doses and thereby prolong activity and also intervals between treatments.

Antibodies of the present invention can be formulated to very high doses which include, but are not limited to 20 mg/ml, 30 mg/ml, 40 mg/ml, 50 mg/ml, 60 mg/ml, 70 mg/ml, 80 mg/ml, 90 mg/ml, or 100 mg/ml. Preferably, antibodies of the present invention can be formulated in a liquid formulation of about 50 mg/ml.

A typical dosage that can be administered to a patient is about 2.5 mg/eye. Typical buffer components that can be used for such a formulation comprise e.g. Sodium Acetate, PS20, and Trehalose Dihydrate.

In one embodiment, the anti-Sema3A antibody is formulated with 10 mM histidine buffer, 240 mM sucrose, 0.02 w/v % polysorbate 20 at pH 5.5 with a final protein concentration of 60 mg/mL.

In some embodiments, the pharmaceutical compositions comprising the anti-Sema3A antibody or an antigen-binding fragment thereof can further comprise a therapeutic agent, either conjugated or unconjugated to the binding agent.

With respect to therapeutic regimens for combinatorial administration, in a specific embodiment, an anti-Sema3A antibody or an antigen-binding fragment thereof is administered concurrently with a therapeutic agent. In another specific embodiment, the therapeutic agent is administered prior or subsequent to administration of the anti-Sema3A antibody or an antigen-binding fragment thereof, by at least an hour and up to several months, for example at least an hour, five hours, 12 hours, a day, a week, a month, or three months, prior or subsequent to administration of the anti-Sema3A antibody or an antigen-binding fragment thereof.

### Method of Treatment

In another aspect, the invention also encompasses any method for treating or preventing a thrombotic disease of the retina, wherein said anti-Sema3A antibody or antigen-binding fragment thereof comprises in a patient in need thereof, said method comprising the administration of an anti-Sema3A antibody of the invention.

Preferably, the invention relates to a method for treating or preventing a thrombotic disease of the retina comprising administering to a patient in need thereof a pharmaceutically effective amount of the antibody according to the invention.

All the disclosed technical features described herein are applicable to said method of treatment.

### Articles of Manufacture

In another aspect, an article of manufacture containing materials useful for the treatment of the disorders described above is included. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port. For example, the container may be an intravenous solution bag or a vial having a stopper pierceable by a hypodermic injection needle. The active agent in the composition is the anti-Sema3A antibody or the antigen-binding fragment thereof. The label on or associated with the container indicates that the composition is used for treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

The invention is further described in the following examples, which are not intended to limit the scope of the invention.

### EXAMPLES

### Example 1: Effects of Anti-Sema3A Antibody in Retinal Vein Occlusion Model Mice

In this study, an exemplary anti-Sema3A antibody according to the invention was evaluated for an intravitreal antibody therapy in retinal ischemia using retinal vein occlusion model of mice. Moreover, to differentiate neutralization of Sema3A/Nrp1 signaling axis from VEGF/Nrp1 axis, monotherapy with anti-Sema3A antibody and its combination with anti-VEGF antibody are also assessed.

### I. Materials

### A. Study Design

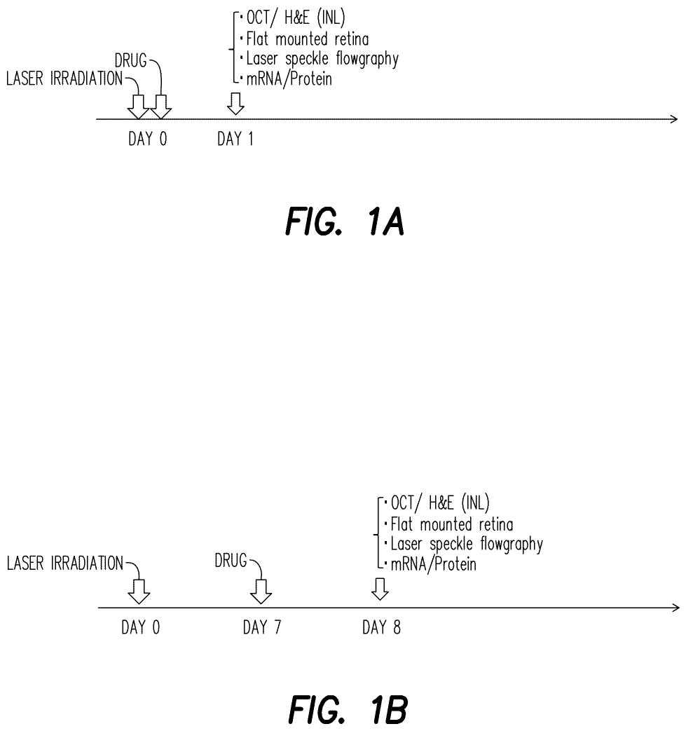

The study design is illustrated in FIGS. 1A and 1B. The present example comprises 4 steps as follows:
Step 1: Edema and damage (Histological analysis, optical coherence tomography (OCT))
Step 2: Blood flow (Laser speckle flowgraphy)
Step 3: Retinal non-perfused area (Fluorescein-stained flat-mounted retina)
Step 4: Protein expression (WB)

### B. Test/Reference Compound

The inventors tested an exemplary antibody according to the invention: clone I. Said antibody comprises a heavy chain comprising the amino acid sequence of SEQ ID NO: 14 and a light chain comprising the amino acid sequence of SEQ ID NO: 15.

The inventors have tested and compared said anti-Sema3A antibody with the commercially available anti-VEGF trap Eylea®.

The compounds were diluted with Avastin buffer (60 mg/ml α,α-trehalose dehydrate, 5.8 mg/ml sodium phosphate (monobasic, monohydrate), 1.2 mg/ml sodium phosphate (dibasic, anhydrous), 0.4 mg/ml polysorbate 20, pH 6.2) to a concentration of 10 mg/mL.

### C. Groups and Protocol

The table 4 below summarises the various groups and type of protocol used for each group.

TABLE 4

| | | Reference/ | Dosage** | Dosing | No. of |
|---|---|---|---|---|---|
| | Induction | Test Reagents | (mg/kg) | Route | animals |
| 1 | Normal | Vehicle | | IVT | 5 × 2 |
| 2 | RVO | Vehicle | | IVT | 5 × 2 |
| 3 | RVO | anti-Sema3A of | 10 μg/eye | IVT | 5 × 2 |
| | | the invention | | | |
| 4 | RVO | anti-VEGF (Eylea ®) | 10 μg/eye | IVT | 5 × 2 |
| 5 | RVO | anti-Sema3A of | 10 μg/eye | IVT | 5 × 2 |
| | | the invention | | | |
| | | anti-VEGF (Eylea ®) | 10 μg/eye | | |

RVO, retinal vein occlusion;
IVT, intravitreal injection

### D. Reagents

The various reagents used are summarized in the following table 5.

TABLE 5

| Name of the Reagent | Vendor | Catalog No. |
|---|---|---|
| Rose Bengal | Wako | 184-00272 |
| Immuno Star ® LD | Wako | 290-69904 |
| Fluorescein conjugated dextran | Sigma-Aldrich | FD2000S-5G |
| di-sodium hydrogenphosphate | Nacalai Tesque | 31723-35 |
| 12-water: Na2HPO3•12H2O | | |
| sodium dihydrogenphosphate | Nacalai Tesque | 31718-15 |
| dihydrate: NaH2PO4•2H2O | | |
| paraformaldehyde | Nacalai Tesque | 162-16065 |
| ketalar | Daiichi Sankyo | GYA0038 |
| | Propharma | |
| xylazine | Bayer Healthcare | KP0C7DJ |
| Hematoxylin 560MX | Leica | 3801575 |
| Alcoholic Eosin Y515 | Leica | 3801615 |
| Potassium chloride | Wako | 160-22115 |
| Sodium chloride | Kishida Chemical | 008-71265 |
| Potassium dihydrogenphosphate | Nacalai Tesque | 28720-65 |

### E. Antibodies for Western Blot Analysis of Protein Expression

Finally, the antibodies used specifically for in vitro analysis of protein expression by Western blot are summarized in the table 6 below.

TABLE 6

| Name/Target | Origin | Vendor | Catalog No. | Lot No. |
|---|---|---|---|---|
| Nrp1 | rabbit | abcam | ab81321 | GR212288-38 |
| TNFα | mouse | Santa Cruz | sc-52746 | J1317 |
| | | Biotechnology | | |
| PlexinA1 | rabbit | abcam | ab23391 | GR285914-16 |
| β actin | mouse | Sigma-Aldrich | A2228 | 067M4856V |

### II. Methods

### A. Animals and RVO Murine Model:

All animal experiments were performed in accordance with the Association for Research in Vision and Ophthalmology (ARVO) statement for the Use of Animals in Ophthalmic and Vision Research, and the experiments were approved and monitored by the Institutional Animal Care and Use Committee of Gifu Pharmaceutical University.

8 weeks-old ddY male mice were obtained from Japan SLC (Shizuoka, Japan) and were housed at 23±3° C., under 12 h light/dark cycles (lights on from 08:00 to 20:00). The mice were anesthetized with a mixture of ketamine (120 mg/kg) and xylazine (6 mg/kg). RVO is developed by laser photocoagulation (532 nm, 50 mW power, 5000 msec duration, 50 μm spot size) of three retinal branch veins of the right eye of each animal following i.v. injection of 8 mg/mL Rose Bengal using an image-guided laser system attached to a Micron IV Retinal Imaging Microscope (Phoenix Research Laboratories, Inc.).

Anti-Sema3A antibody of the invention and/or anti-VEGF trap was intravitreally injected immediately or 7 days after laser irradiation into the right eye of each mouse at a dosage of 10 μg/eye and an injection volume of 2 μL.

### B. Histology

Hematoxylin and eosin stain (H&E) staining was performed to visualize histological changes of mouse eye sections. Eyes used for histological analysis were kept immersed for at least 48 h at 4° C. in 4% paraformaldehyde (PFA). Six paraffin-embedded sections (5 μm) were cut through the optic disc of each eye and stained with hematoxylin and eosin. Images were photographed with a fluorescence microscope (BZ-710; Keyence). The thickness of the inner nuclear layer (INL) from the optic disc was measured on the photographs every 240 μm from the optic disc toward the periphery with Image J (National Institutes of Health, Bethesda). The data from three sections selected randomly from the six sections were averaged for each eye.

C. Blood Flow Measurement with Laser Speckle Flowgraphy:

The mean blur rate (MBR) images, an index of the relative blood flow velocity, were acquired continuously using a laser speckle flowgraphy device (LSFG; Softcare) at a rate of 30 frames per second over a time period of approximately 4 s. The measured fundus area was approximately 3.8×3 mm (width×height) with an estimated tissue penetration of 0.5-1 mm. After the image acquisition, the vessel and tissue areas on the optic nerve head area were automatically detected by the LSFG Analyzer software (version 3.1.14.0; Software Co., Ltd.) using the so-called vessel extraction function.

### D. Imaging of Retinal Non-Perfused Area:

The mice were injected with 0.5 mL of 20 mg/mL fluorescein conjugated dextran dissolved in PBS into the tail veins before the sampling. Eyes were enucleated and fixed for 7 h in 4% PFA, and retinal flat-mounts were prepared. Images of the retinal flat-mounts were taken with Metamorph (Universal Imaging Corp) and analysed using ImageJ processing software to determine the size of the retinal non-perfused areas.

### E. Western Blot Analysis for Protein Expression

Western blot analysis was performed by a standard method. The immunoreactive bands were made visible by Immuno Star® LD, and their densities were measured with the LAS-4000 Luminescent Image Analyzer (Fuji Film Co. Ltd.). For quantitative analysis, the total protein signals were used as the loading controls for the phosphoprotein signals.

### III. Results

### A. The Anti-Sema3A Antibody of the Invention Ameliorates Cystoid Edema and the Retinal Thinning in Inner Nuclear Layer of RVO Murine Model.

The inventors investigated whether the cystoid edema induced by RVO can be ameliorated by the administration of anti-Sema3A antibody of the invention.

The treatment protocol for this experiment comprises an early phase administration after laser irradiation and a late phase administration at 7 days after the laser irradiation. Basically, the anti-Sema3A antibody and/or anti-VEGF trap is intravitreally injected either immediately or 7 days after laser irradiation into the right eye of each mouse at a dosage of 10 μg/eye.

The thickness of the retinal thinning of the inner nuclear layer (INL) was markedly increased 1 day after the laser irradiation, and this increase was suppressed by the administration of the anti-Sema3A antibody of the invention. The combination of the antibody of the invention with an anti-VEGF trap as well as the anti-VEGF trap alone achieved the same effect in this early phase after RVO induction.

To examine the effect of anti-Sema3A antibody on the retinal thinning of the inner nuclear layer (INL) at a late phase after RVO induction, the mice were intravitreally injected with anti-Sema3A antibody of the invention and/or an anti-VEGF trap at 7 days after laser irradiation.

The thickness of INL was significantly decreased at 8 days after the laser irradiation in the vehicle-treated group. The administration of an anti-VEGF trap increased the degree of the retinal thinning. However, the retinal thinning was suppressed by the intravitreal injection of anti-Sema3A antibody of the invention at 7 days after the laser irradiation.

The results (FIGS. 1A and 1B) show that the antibody of the invention suppresses the retinal thinning, corroborating its beneficial use in the treatment of thrombotic disease of the retina such as RVO. The results also differentiate the antibody of the invention from a treatment with an anti-VEGF trap since the latter does not achieve beneficial effects in all phases after RVO induction.

### B. Ocular Blood Flow is Improved by the Administration of Anti-Sema3A Antibody of the Invention in RVO Murine Model.

The inventors examined the changes in the ocular blood flow at 1 or 8 days after the laser irradiation by the anti-Sema3A antibody of the invention with laser speckle flowgraphy.

The treatment protocol for this experiment comprises an early phase administration after laser irradiation (FIG. 1A) and a late phase administration at 7 days after the laser irradiation (FIG. 1B). Basically, the anti-Sema3A antibody and/or anti-VEGF trap is intravitreally injected either immediately or 7 days after laser irradiation into the right eye of each mouse at a dosage of 10 μg/eye.

The inventors have shown that the blood flow was significantly reduced at 1 day after the laser irradiation in the vehicle-treated group.

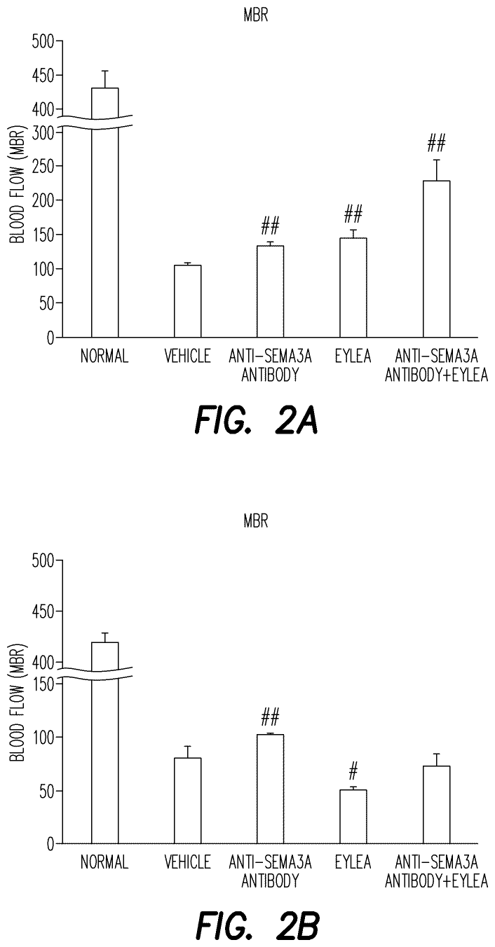

With an early administration after laser irradiation, the decrease of the blood flow was reduced on day 1 after the administration of anti-Sema3A antibody of the invention and the administration. Administration of the anti-VEGF trap achieved the same effect on blood flow. The combination of the anti-Sema3A antibody of the invention and the anti-VEGF trap immediately after the laser irradiation resulted in a more pronounced reduction of the decrease in blood flow in this early phase after RVO induction (FIG. 2A).

The inventors investigated the ocular blood flow with anti-Sema3A antibody and/or anti-VEGF trap at 7 days after laser irradiation into the right eye of each mouse at a dosage of 10 μg/eye (late phase administration after laser irradiation). The blood flow was significantly reduced at 8 days after the laser irradiation in the vehicle-treated group.

The results show that the injection of anti-VEGF trap increased the degree of reduction of the retinal blood flow. On the contrary, the blood flow on the administration of anti-Sema3A antibody of the invention was significantly better than vehicle-treated group. Combination of the anti-Sema3A antibody of the invention with an anti-VEGF trap at this late phase after RVO induction neutralized the beneficial effect of the anti-Sema3A antibody of the invention (FIG. 2B).

These data show that the antibody of the invention significantly improves the blood flow at all phases after RVO induction. They further show the superiority of the antibody of the invention in improving the blood flow when compared to the therapeutic strategy based on anti-VEGF alone.

### C. Anti-Sema3A Antibody of the Invention Reduces the Size of Retinal Non-Perfused Areas in RVO Murine Model.

To investigate the effect of anti-Sema3A antibody on the size of the non-perfused areas, the inventors injected intravitreally either immediately or 7 days after the laser irradiation an anti-Sema3A antibody according to the invention and/or an anti-VEGF trap.

The administration of anti-Sema3A antibody immediately after the laser irradiation led to a significant reduction in the size of the non-perfused areas at 1 day after the laser irradiation compared to vehicle-treated group. Administration of an anti-VEGF trap or a combination of the anti-Sema3A antibody of the invention with an anti-VEGF trap achieved about the same effect.

Regarding the late phase administration after laser irradiation, the inventors have shown that the size of non-perfused areas was increased by the administration of anti-VEGF trap at 7 days after the laser irradiation. On the contrary, the inventors have shown that the administration of anti-Sema3A antibody of the invention at 7 days after the laser irradiation led to a reduction of the size of non-perfused areas compared to the vehicle-treated group. Administration of a combination of the anti-Sema3A antibody of the invention with the anti-VEGF trap neutralized the beneficial effect of the anti-Sema3A antibody of the invention.

These results indicate that the anti-Sema3A antibody of the invention reduces the size of non-perfused areas after 7 days, in a better extend than strategy based on the therapeutic use of an anti-VEGF trap. This corroborates the beneficial use of the antibody of the invention in the treatment of a thrombotic diseases such as RVO.

### D. The Expression of TNF-α and Sema3A Related Receptor (Plexin A1 and Neuropilin1) is Decreased by Anti-Sema3A Antibody in RVO Murine Model.

The protein expression of TNF-α and Sema3A related receptor (Plexin A1 and Neuropilin1) was investigated.

The expressions of TNF-α and Sema3A related receptor components (PlexinA1 or Neuropillin1) were determined after intravitreal injection of anti-Sema3A antibody of the invention and/or anti-VEGF trap either immediately or 7 days after the laser irradiation.

Expressions of TNF-α and PlexinA1 were both increased in the vehicle-treated group at 1 day after the laser irradiation. The early injection of the anti-Sema3A antibody of the strongly reduced the levels of expressions compared to vehicle-treated group. The combination of the anti-Sema3A antibody of the invention with an anti-VEGF trap achieved the same effect. While an anti-VEGF trap alone also reduced TNF-α in this early phase after RVO induction, it did not significantly affect the expression of PlexinA1.

TNF-α and neuropilin1 were increased in the vehicle-treated group at 8 days after the laser irradiation. The administration of anti-Sema3A antibody of the invention however reduced those factors in late phase. On the other hand, the anti-VEGF trap injection did not affect Nrp1 expression and increased TNF-α compared to vehicle-treated group in this late phase after RVO induction. Combination of the anti-Sema3A antibody of the invention with an anti-VEGF trap attenuated the effects of the anti-Sema3A antibody of the invention.

### IV. Conclusion

Overall, these data show that the expression levels of Sema3A related receptor and inflammatory factors were increased in the eyes with RVO.

Injection of anti-Sema3A antibody according to the invention at an early phase after RVO induction significantly reduced the retinal edema, the size of non-perfused areas and the decrease of blood flow. Furthermore, the increased expression of TNF-α and Sema3A related receptor (PlexinA1) was reduced.

In addition, injection of anti-Sema3A antibody at a late phase after RVO induction also improved those pathological symptoms and the increased expression of TNF-α and Sema3A related receptor (Neuropilin1) was reduced.

The downregulation of TNF-α and the Sema3A related receptor (Neuropilin1 and PlexinA1) may have contributed to the amelioration of the pathological symptoms in the RVO murine model after administration of an anti-Sema3A antibody of the invention.

The present data confirm that the antibody of the invention is highly promising for treating patients suffering from a thrombotic disease of the retina, especially RVO. In particular, the anti-Sema3A antibody of the invention shows beneficial effects in all phases after RVO induction which differentiates it from an anti-VEGF trap.

### Example 2: Affinity and Cellular Potency

### A) Affinity

The running buffer for this experiment and all dilutions (except where stated) were done in PBS-T-EDTA with 0.01% Tween20 [100 ul of 100% Tween20 was added to 2 L of PBS-T-EDTA to make final Tween 20 concentration of 0.01%]. The GLM sensorchip was normalized and pre-conditioned as per the manufacturer's recommendations. The sensorchip was activated with equal mixture of EDC/s-NHS in the horizontal direction for 300 sec at a flow rate of 30 μl/min and immobilized with Human Fab Binder (10 μg/ml in 10 mM acetate pH 5.0) in the horizontal direction for 300 sec at a flowrate of 30 μl/min resulting in ˜ 6739-7414 RU of Human Fab Binder on the surface. The sensorchip was deactivated with 1M ethanolamine HCl in the horizontal direction for 300 sec at a flowrate of 30 μl/min. The sensorchip was stabilized with 18 sec of 10 mM glycine, pH 2.1 at a flowrate of 100 μl/min 1 time horizontally and 1 time vertically.

The inventors tested an exemplary antibody according to the invention (clone I). Said antibody (0.5 μg/ml) was captured on the Human Fab Binder surface vertically for 300 sec at a flowrate of 25 μl/min resulting ˜180 RU capture level. The baseline was stabilized by injecting PBS-T-EDTA for 60 sec at a flowrate of 40 μl/min horizontally. The analyte was injected horizontally over the captured antibody for 600 sec at a flowrate of 40 μl/min and a dissociation for 7200 sec. The concentrations of the analytes were 0 nM, 0.625 nM, 1.25 nM, 2.5 nM, 5 nM, and 10 nM. The surface was regenerated by injecting 10 mM glycine, pH 2.1 for 18 sec at a flowrate of 100 μl/min one time horizontally and one time vertically. PBS-T-EDTA was injected for 60 sec at a flowrate of 25 l/min one time vertically.

The interspot (interactions with sensor surface) and blank (PBS-T-EDTA with 0.01% Tween20 or 0 nM analyte) were subtracted from the raw data. Sensorgrams were then fit globally to 1:1 Langmuir binding to provide on-rate (ka), off-rate (kd), and affinity (K_{D}) values.

### B) Cellular Potency

For determination of a functional potency in the cytoskeletal collapse assay, Sema3A concentration response curves were combined with increasing concentrations of antibody as IC₅₀ shift experiments. A Gaddum Schild plot was performed to calculate the pA2 value (the negative logarithm of the concentration of antibody needed to shift the Sema3A concentration response curve by factor 2). The potency in pM was calculated from the pA2 value as =POTENCY(10;−X).

The results are summarised in the table 7 below.

TABLE 7

| | | | | | | Functional antagonism in |
|---|---|---|---|---|---|---|
| | Affinity (K_{d}) [pM] | | | | | cytoskeletal collapse assay (A₂) [pM] |
| Molecule | Human | Cyno | Mouse | Rat | Rabbit | Human |
| Antibody of | 29 | 28 | 27 | 27 | 42 | 69 |
| the invention | | | | | | |
| (clone I) | | | | | | |

### Example 3: Assessment of the Immunogenicity of the Antibody of the Invention

The inventors have assessed the predicted immunogenicity of an exemplary antibody according to the invention, clone I. Said antibody comprises a heavy chain and a light chain comprising the amino acid sequences of SEQ ID NO: 14 and SEQ ID NO: 15 respectively.

For this purpose, they have used an in silico tool for predicting T cell epitopes (EpiMatrix developed by EpiVax).

By screening the sequences of many human antibody isolates, EpiVax has identified several highly conserved HLA ligands which are believed to have a regulatory potential. Experimental evidence suggests many of these peptides are, in fact, actively tolerogenic in most subjects. These highly conserved, regulatory, and promiscuous T cell epitopes are now known as Tregitopes (De Groot et al. Blood. 2008 Oct. 15; 112(8):3303-11). The immunogenic potential of neo-epitopes contained in humanized antibodies can be effectively controlled in the presence of significant numbers of Tregitopes.

For the purposes of antibody immunogenicity analysis, EpiVax has developed a Tregitope-adjusted EpiMatrix Score and corresponding prediction of anti-therapeutic antibody response. To calculate the Tregitope-adjusted EpiMatrix Score, the scores of the Tregitopes are deducted from the EpiMatrix Protein Score. The Tregitope-adjusted scores have been shown to be well correlated with observed clinical immune response for a set of 23 commercial antibodies (De Groot et al. Clin Immunol. 2009 May; 131(2):189-201).

The results on the EpiMatrix scale are summarised in the table 8 below.

TABLE 8

| | Heavy Chain | | | | Light chain | |
|---|---|---|---|---|---|---|
| | (% human) | | Epivax | Epivax | (% human) | |
| Molecule | FR | V-gene | (VH) | (Vκ) | FR | V-gene |
| Antibody of | 97 | 91 | −27.27 | −21.79 | 98 | 88 |
| the invention | | | | | | |
| (clone I) | | | | | | |

Sequences of the antibody of the invention score on the low end of EpiMatrix scale, indicating that the antibody of the invention has a strongly limited potential for immunogenicity. Said EpiMatrix scale is well known by the person skilled in the art and can be found inter alia in FIG. 2 of the publication Mufarrege et al. Clin Immunol. 2017 March; 176:31-41.

### Example 4: Comparison of Binding Affinity Between the Antibody of the Invention and Chiome Antibody

For comparison purposes, the inventors have developed the humanized antibody directed against Sema3A disclosed in WO2014123186 (Chiome Bioscience) with the following features:
the heavy chain is as shown in SEQ ID NO: 11 in WO2014123186, and
the light chain is as shown in SEQ ID NO: 12 in WO2014123186.

The inventors have developed 2 forms of this antibody:
one formatted on IgG1KO Fc, referred to in the followings as “Chiome antibody A” and
one formatted on IgG1KO-FcRn null referred to in the followings as “Chiome antibody B”.

A high surface density of anti-human Fab antibody (GE Healthcare) was immobilized over a GLM chip (BioRad) via direct amine coupling over 6 horizontal channels according to the BioRad manufacturer's manual.

The antibody of the invention (clone I) and the Chiome antibodies were captured over the anti-human Fab antibody surface over 5 of 6 vertical channels with a minimum surface density for the kinetic binding assay. Human Sema3A was prepared in PBS-T-EDTA buffer (BioRad) at concentrations of 100, 50, 25, 12.5, 10, 6.25, 5, 2.5, 1.25, 0.625 and 0 nM. A PBS-T-EDTA buffer injection was used as a double reference for the kinetic data analysis. Each of the human Sema3A solutions and PBS-T-EDTA buffer were injected simultaneously over the 6 horizontal channels for 10 min at a flow rate of 40 μL/min followed by 2 hr dissociation phase. The surfaces were regenerated by an 18 sec injection of 10 mM pH 2.1 glycine HCl (GE Healthcare) at a flow rate of 100 μL/min followed by an injection of 60 sec PBS-T-EDTA at a flow rate of 25 μL/min. The binding sensorgrams were fit to 1:1 langmuir model to calculate on-rate, off-rate, and affinity.

The Kinetic and affinity data of the antibody of the invention and the Chiome antibodies binding to human Sema3A are listed in table 9 below.

TABLE 9

| Sample Name | KD to HuSema3A |
|---|---|
| Chiome Antibody A | 56.4 nM |
| Chiome Antibody B | 55.9 nM |
| Antibody of the invention (clone I) | 32.0 pM |

### Conclusion

The results show that the antibody of the invention proved to have superior binding affinity to human Sema3A than the prior art antibodies disclosed in WO2014123186 (Chiome Bioscience).

### Example 5: Comparison of Binding Affinity Between the Antibody of the Invention and Samsung scFv

scFv fragments as disclosed in WO2017074013 (Samsung) have been compared.

For comparison purposes, the inventors have developed 3 disclosed scFv fragments (“Samsung scFv”) with the features disclosed in the table 10 below.

TABLE 10

| | | SEQ ID NO as set forth in |
|---|---|---|
| Name of the antibody | Sequences | WO2017074013 |
| Samsung scFv 1 | Heavy chain | 19 |
| | Light chain | 20 |
| Samsung scFv 2 | Heavy chain | 21 |
| | Light chain | 22 |
| Samsung scFv 3 | Heavy chain | 23 |
| | Light chain | 24 |

A high surface density of anti-His antibody (GE Healthcare) was immobilized over a GLM chip (BioRad) via direct amine coupling over 6 horizontal channels according to the BioRad manufacturer's manual. The Samsung scFv antibodies were captured over the anti-His antibody surface over 5 of 6 vertical channels with a minimum surface density for the kinetic binding assay. Human Sema3A was prepared in PBS-T-EDTA buffer (BioRad) at concentrations of 100, 50, 25, 12.5, 10, 6.25, 5, 2.5, 1.25, 0.625 and 0 nM. A PBS-T-EDTA buffer injection was used as a double reference for the kinetic data analysis. Each of the human Sema3A solutions and PBS-T-EDTA buffer were injected simultaneously over the 6 horizontal channels for 10 min at a flow rate of 40 L/min followed by 1 hr dissociation phase. The surfaces were regenerated by an 18 sec injection of 10 mM pH 2.1 glycine HCl (GE Healthcare) at a flow rate of 100 μL/min followed by an injection of 60 sec PBS-T-EDTA at a flow rate of 25 μL/min. The binding sensorgrams were fit to 1:1 langmuir model to calculate on-rate, off-rate, and affinity.

The binding for the antibody of the invention to human Sema3A (clone I) was done using similar method but goat anti-human IgG (Invitrogen) was used to capture the antibody of the invention. Binding of the antibody of the invention and Samsung ScFv to Cynomology, mouse, rat, or rabbit Sema3A was also done using the same methods.

The Kinetic and affinity data of the antibody of the invention and the Samsung scFv are listed in the table 11 below.

TABLE 11

| | K_{D} to | K_{D} to | K_{D} to | K_{D} to | K_{D} to |
|---|---|---|---|---|---|
| Name | Hu- | Cyno- | Mouse- | Rat- | Rabbit- |
| of the | Sema3A | Sema3A | Sema3A | Sema3A | Sema3A |
| antibody | (pM) | (pM) | (pM) | (pM) | (pM) |
| Samsung | 359 | 89.0 | 105 | <20 | 112 |
| scFv 1 | | | | | |
| Samsung | 359 | 118 | 117 | <20 | 122 |
| scFv 2 | | | | | |
| Samsung | 296 | 68.0 | 88.8 | <20 | 59.5 |
| scFv 3 | | | | | |
| Antibody | 34.7 | 35.0 | 35.0 | 23.5 | 40.1 |
| of the | | | | | |
| invention | | | | | |
| (clone I) | | | | | |

### Conclusion

The antibody of the invention has a higher binding affinity to human, cyno, mouse, or rabbit Sema3A than the 3 Samsung scFv as disclosed in WO2017074013.

1-15. (canceled)

16. A method for treating a retinal vein occlusion (RVO), comprising administering a therapeutically effective amount of an anti-Sema3A antibody or an antigen-binding fragment thereof to a patient in need thereof, wherein the antibody or antigen-binding fragment thereof comprises a heavy chain variable region comprising a heavy chain complementarity determining region (CDR) 1 having the amino acid sequence of SEQ ID NO: 1 (H-CDR1), a heavy chain CDR2 having the amino acid sequence of SEQ ID NO: 2 (H-CDR2), and a heavy chain CDR3 having the amino acid sequence of SEQ ID NO: 3 (H-CDR3); and a light chain variable region comprising a light chain CDR1 having the amino acid sequence of SEQ ID NO: 4 (L-CDR1), a light chain CDR2 having the amino acid sequence of SEQ ID NO: 5 (L-CDR2), and a light chain CDR3 having the amino acid sequence of SEQ ID NO: 6 (L-CDR3).

17. The method according to claim 16, wherein the RVO comprises central retinal vein occlusion (CRVO).

18. The method according to claim 16, wherein the RVO comprises hemispheric retinal vein occlusion (HRVO).

19. The method according to claim 16, wherein the RVO comprises branch retinal vein occlusion (BRVO).

20. The method of claim 16, wherein said method ameliorates cystoid edema in said patient.

21. The method of claim 16, wherein said method ameliorates retinal thinning in the inner nuclear layer in said patient.

22. The method of claim 16, wherein said method improves ocular blood flow in said patient.

23. The method of claim 16, wherein said method reduces the size of one or more retinal non-perfused areas in said patient.

24. The method of claim 16, wherein said method decreases the expression of TNF-α in said patient.

25. The method of claim 16, wherein said method decreases the expression of Plexin A1 in said patient.

26. The method of claim 16, wherein said method decreases the expression of Neuropilin-1 in said patient.

27. The method according to claim 16, wherein the anti-Sema3A antibody or the antigen-binding fragment thereof comprises a heavy chain variable region comprising an amino acid sequence at least 95% identical to the amino acid sequence of SEQ ID NO: 7, SEQ ID NO: 8, SEQ ID NO: 9 or SEQ ID NO: 10; and a light chain variable region comprising an amino acid sequence at least 95% identical to the amino acid sequence of SEQ ID NO: 11, SEQ ID NO: 12 or SEQ ID NO: 13.

28. The method according to claim 16, wherein the antibody or the antigen-binding fragment thereof comprises a heavy chain variable region comprising the amino acid sequence of SEQ ID NO: 7, SEQ ID NO: 8, SEQ ID NO: 9 or SEQ ID NO: 10; and a light chain variable region comprising the amino acid sequence of SEQ ID NO: 11, SEQ ID NO: 12 or SEQ ID NO: 13.

29. The method according to claim 16, wherein the antibody or the antigen-binding fragment thereof comprises:
a. a variable heavy chain comprising the amino acid sequence of SEQ ID NO: 7 and a variable light chain comprising the amino acid of SEQ ID NO: 11;
b. a variable heavy chain comprising the amino acid sequence of SEQ ID NO: 8 and a variable light chain comprising the amino acid sequence of SEQ ID NO: 11;
c. a variable heavy chain comprising the amino acid sequence of SEQ ID NO: 9 and a variable light chain comprising the amino acid sequence of SEQ ID NO: 12; or
d. a variable heavy chain comprising the amino acid sequence of SEQ ID NO: 10 and a variable light chain comprising the amino acid sequence of SEQ ID NO: 13.

30. The method according to claim 16, wherein the antibody or the antigen-binding fragment thereof comprises a heavy chain comprising the amino acid sequence of SEQ ID NO: 14, SEQ ID NO: 16, SEQ ID NO: 17 or SEQ ID NO: 19; and a light chain comprising the amino acid sequence of SEQ ID NO: 15, SEQ ID NO: 18 or SEQ ID NO: 20.

31. The method according to claim 16, wherein the antibody or the antigen-binding fragment thereof comprises a heavy chain having an amino acid sequence consisting of the amino acid sequence of SEQ ID NO: 14, SEQ ID NO: 16, SEQ ID NO: 17 or SEQ ID NO: 19; and a light chain having an amino acid sequence consisting of the amino acid sequence of SEQ ID NO: 15, SEQ ID NO: 18 or SEQ ID NO: 20.

32. The method according to claim 16, wherein the antibody or the antigen-binding fragment thereof comprises:
a. a heavy chain comprising the amino acid sequence of SEQ ID NO: 14 and a light chain comprising the amino acid sequence of SEQ ID NO: 15;
b. a heavy chain comprising the amino acid sequence of SEQ ID NO: 16 and a light chain comprising the amino acid sequence of SEQ ID NO: 15;
c. a heavy chain comprising the amino acid sequence of SEQ ID NO: 17 and a light chain comprising the amino acid sequence of SEQ ID NO: 18; or
d. a heavy chain comprising the amino acid sequence of SEQ ID NO: 19 and a light chain comprising the amino acid sequence of SEQ ID NO: 20.

33. The method according to claim 16, wherein said patient is a human.

34. The method according to claim 16, wherein the antibody or an antigen-binding fragment thereof is administered by a parenteral route, intravenous route, intravitreal route or subcutaneous route of administration.

35. The method according to claim 16, wherein the antibody or an antigen-binding fragment thereof is administered by an intravitreal route of administration.

36. A method for treating retinal vein occlusion (RVO), comprising administering a therapeutically effective amount of an anti-Sema3A antibody or an antigen-binding fragment thereof to a patient in need thereof, wherein the antibody or fragment thereof binds to at least one amino acid residue within amino acid regions 370-382 of human Sema3A as set forth in SEQ ID NO: 22.
